(19) United States
(12) Patent Application Publication
(10) Pub. No.: US 2025/0325670 A1
(43) Pub. Date: Oct. 23, 2025

(54) NOVEL BORON AGENT

(71) Applicants: TOKYO INSTITUTE OF TECHNOLOGY, Tokyo (JP); EDUCATIONAL FOUNDATION OF OSAKA MEDICAL AND PHARMACEUTICAL UNIVERSITY, Takatsuki-shi, Osaka (JP); STELLA PHARMA CORPORATION, Osaka-shi, Osaka (JP)

(72) Inventors: Hiroyuki NAKAMURA, Tokyo (JP); Taiki MORITA, Tokyo (JP); Satoshi OKADA, Tokyo (JP); Kai NISHIMURA, Tokyo (JP); Shinji KAWABATA, Takatsuki-shi (JP); Masahiko WANIBUCHI, Takatsuki-shi (JP); Yusuke FUKUO, Takatsuki-shi (JP); Hideki KASHIWAGI, Takatsuki-shi (JP)

(73) Assignees: TOKYO INSTITUTE OF TECHNOLOGY, Tokyo (JP); EDUCATIONAL FOUNDATION OF OSAKA MEDICAL AND PHARMACEUTICAL UNIVERSITY, Takatsuki-shi, Osaka (JP); STELLA PHARMA CORPORATION, Osaka-shi, Osaka (JP)

(21) Appl. No.: 18/281,507

(22) PCT Filed: Mar. 10, 2022
(86) PCT No.: PCT/JP2022/010452

§ 371 Date: Mar. 6, 2024

(30) Foreign Application Priority Data
Mar. 12, 2021 (JP) 2021-039885
Jun. 9, 2021 (JP) 2021-096432

(51) Int. Cl. A61K 41/00; A61K 51/04

(52) U.S. Cl. CPC A61K 41/0095; A61K 51/0497

(57) ABSTRACT

Provided is a boron agent for boron neutron capture therapy, comprising a compound represented by the following formula (I) or (II):

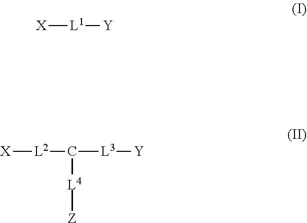

wherein C represents a carbon atom, L¹, L², L³, and L⁴ each independently represent a divalent group that functions as a spacer, X represents a group that binds to albumin, Y represents a group containing ¹⁰B, and Z represents a group that binds to a folate receptor.

### TECHNICAL FIELD

The present invention relates to a boron agent for boron neutron capture therapy, and an imaging agent for PET (positron emission tomography) for predicting an effect of the boron agent for boron neutron capture therapy.

### BACKGROUND ART

Boron neutron capture therapy (BNCT) uses a powerful particle beam generated by the nuclear reaction of a low-energy thermal or epithermal neutron with boron, and is attracting attention as an ultimate minimally invasive treatment method. BPA, a preceding agent for BNCT, was approved for “unresectable locally advanced or locally recurrent head and neck cancer” in March 2020, along with a domestic compact accelerator for BNCT, and treatment covered by insurance began in June. The only boron agent used in BNCT is BPA, but approximately 40% of patients targeted for BNCT treatment are insensitive to BPA, and many patients cannot undergo BNCT, and thus the development of a new agent is urgently needed.

As candidates for a boron agent to replace BPA, the present inventors have developed a conjugate in which a boron cluster and albumin are bound via maleimide (Patent Literature 1), a folic acid derivative that targets a folate receptor highly expressed in many cancer cells (Patent Literature 2), and the like.

### CITATION LIST

### Patent Literature

[Patent Literature 1] International Publication No. WO 2017/026276
[Patent Literature 2] Japanese Patent Laid-Open No. 2019-38778

### SUMMARY OF INVENTION

### Technical Problem

The above boron agent developed by the present inventors can be expected to have a high antitumor effect, but the development of a new boron agent is desired for more effective cancer treatment. The present invention has been made under such a background, and an object of the present invention is to provide a novel boron agent for BNCT.

In addition, before administering the above agent for BNCT to a patient, administering the agent for BNCT labeled with ¹⁸F and examining the patient with PET allows prediction of the effect of the agent. Another object of the present invention is to provide such an agent for PET.

### Solution to Problem

The present inventors have carried out intensive studies in order to solve the above problems and as a result found that the antitumor effect of a boron agent can be improved by binding boron to a ligand for albumin. In addition, the present inventors have found that the antitumor effect of the boron agent can be further improved by binding the boron agent to a folate receptor recognition site. The present invention has been completed based on these findings.

That is, the present invention provides the following (1) to (12).

(1) A boron agent for boron neutron capture therapy, comprising a compound represented by the following formula (I) or (II):

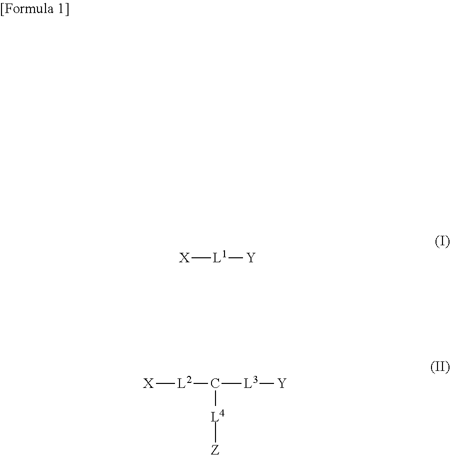

wherein C represents a carbon atom, L¹, L², L³, and L⁴ each independently represent a divalent group that functions as a spacer, X represents a group that binds to albumin, Y represents a group containing ¹⁰B, and Z represents a group that binds to a folate receptor.

(2) The boron agent for boron neutron capture therapy according to (1), wherein L¹, L², L³, and L⁴ in formulas (I) and (II) are each an alkylene group, provided that one or more —CH₂— of the alkylene group may optionally be substituted with —O—, —S—, —NH—, or —CO—.

(3) The boron agent for boron neutron capture therapy according to (1) or (2), wherein X in formulas (I) and (II) is a group represented by any of the following formulas (A) to (C):

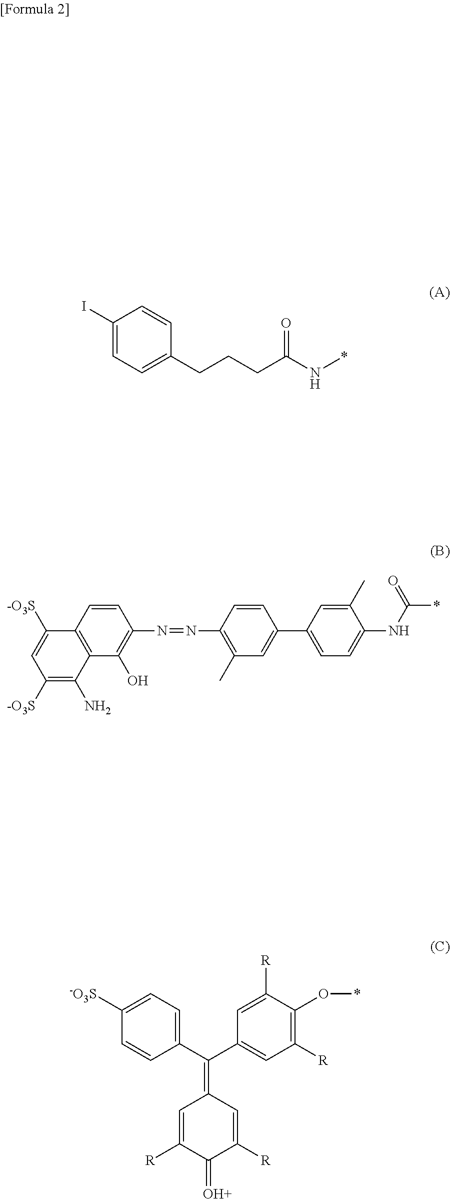

wherein * represents a point of attachment, and R represents a hydrogen atom, a fluorine atom, a chlorine atom, a bromine atom, or an iodine atom.

(4) The boron agent for boron neutron capture therapy according to any one of (1) to (3), wherein Y in formulas (I) and (II) is a group derived from a boron cluster.

(5) The boron agent for boron neutron capture therapy according to any one of (1) to (4), wherein Z in formula (II) is a group represented by the following formula (D):

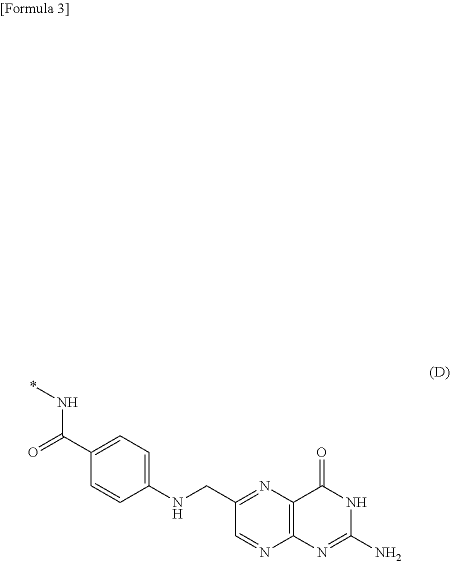 [SMILES: *NC(=O)c1ccc(NCc2cnc3nc(N)[nH]c(=O)c3n2)cc1]

wherein * represents a point of attachment.

(6) The boron agent for boron neutron capture therapy according to (1), wherein the compounds represented by formulas (I) and (II) are compounds represented by the following formulas (Ia) and (IIa), respectively:

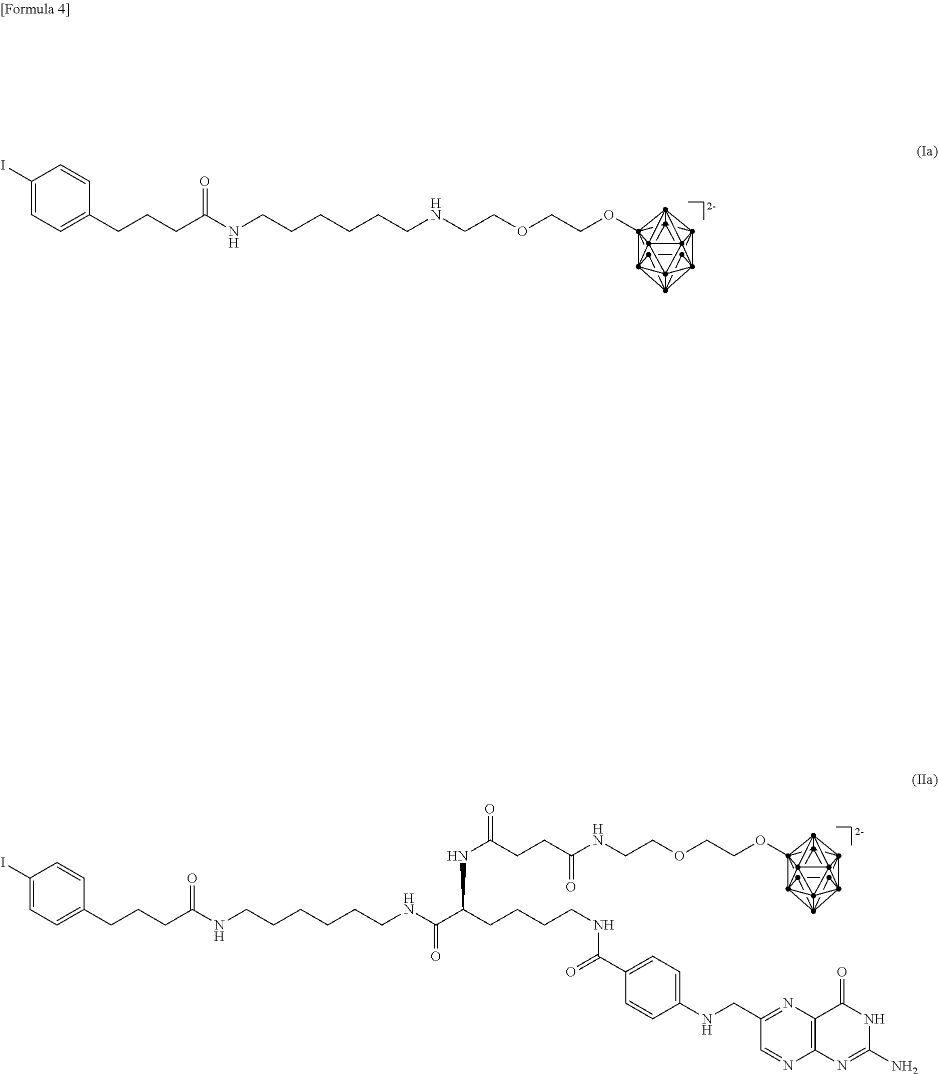

(7) An imaging agent for PET, comprising a compound represented by the following formula (III) or (IV):

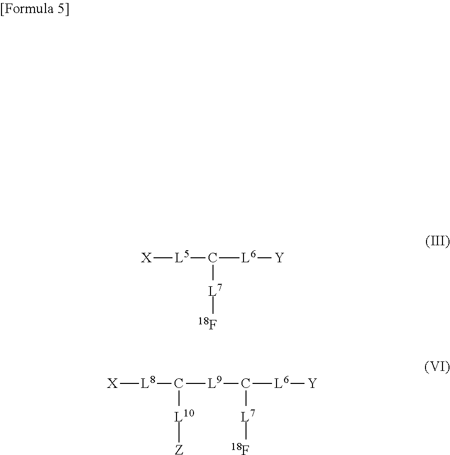

wherein C represents a carbon atom, ¹⁸F represents a radioactive fluorine atom having a mass number of 18, L⁵, L⁶, L⁷, L⁸, L′, and L¹⁰ each independently represent a divalent group that functions as a spacer, X represents a group that binds to albumin, Y represents a group containing ¹⁰B, and Z represents a group that binds to a folate receptor.

(8) The imaging agent for PET according to (7), wherein L⁵, L⁶, L⁸, L⁹, and L¹⁰ in formulas (III) and (IV) are each an alkylene group, provided that one or more —CH₂— of the alkylene group may optionally be substituted with —O—, —S—, —NH—, or —CO—, and L⁷ is an alkylene group, provided that one or more —CH₂— of the alkylene group may optionally be substituted with —O—, —S—, —NH—, or —CO—, and one —CH₂— of the alkylene group may optionally be substituted with a divalent group formed by a click reaction.

(9) The imaging agent for PET according to (7) or (8), wherein X in formulas (III) and (IV) is a group represented by any of the following formulas (A) to (C):

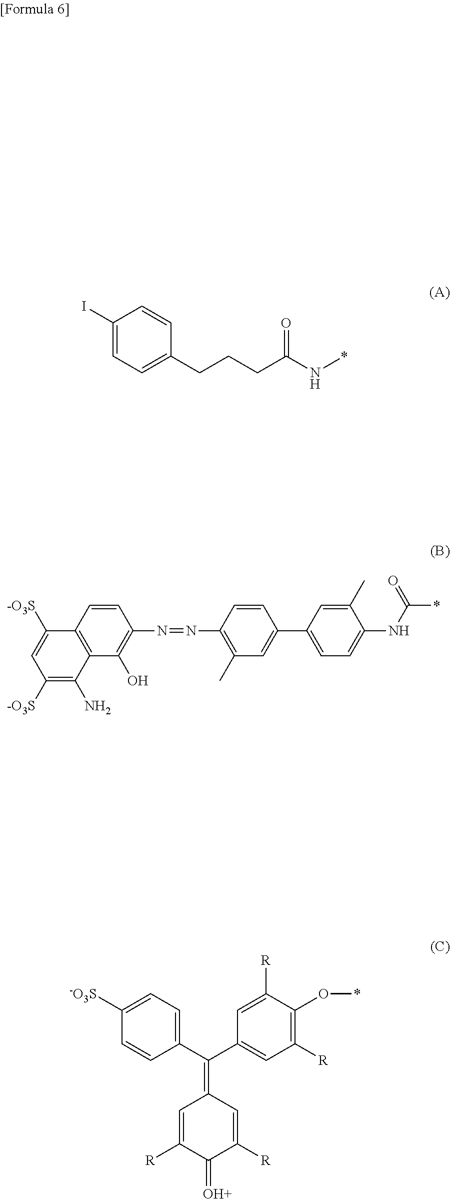

wherein * represents a point of attachment, and R represents a hydrogen atom, a fluorine atom, a chlorine atom, a bromine atom, or an iodine atom.

(10) The imaging agent for PET according to any one of (7) to (9), wherein Y in formulas (III) and (IV) is a group derived from a boron cluster.

(11) The imaging agent for PET according to any one of (7) to (10), wherein Z in formula (IV) is a group represented by the following formula (D):

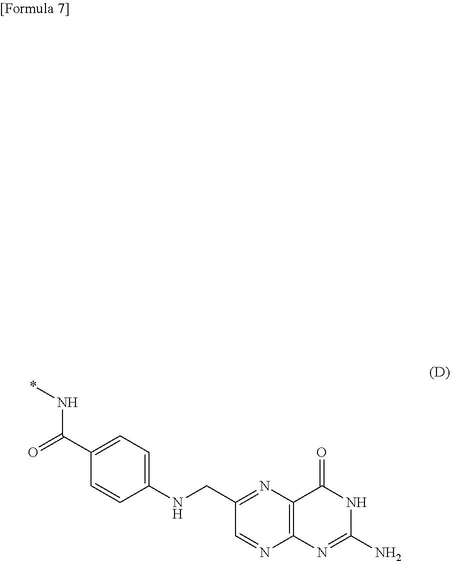 [SMILES: *NC(=O)c1ccc(NCc2cnc3nc(N)[nH]c(=O)c3n2)cc1]

wherein * represents a point of attachment.

(12) The imaging agent for PET according to (7), wherein the compound represented by formula (IV) is a compound represented by the following formula (IVa):

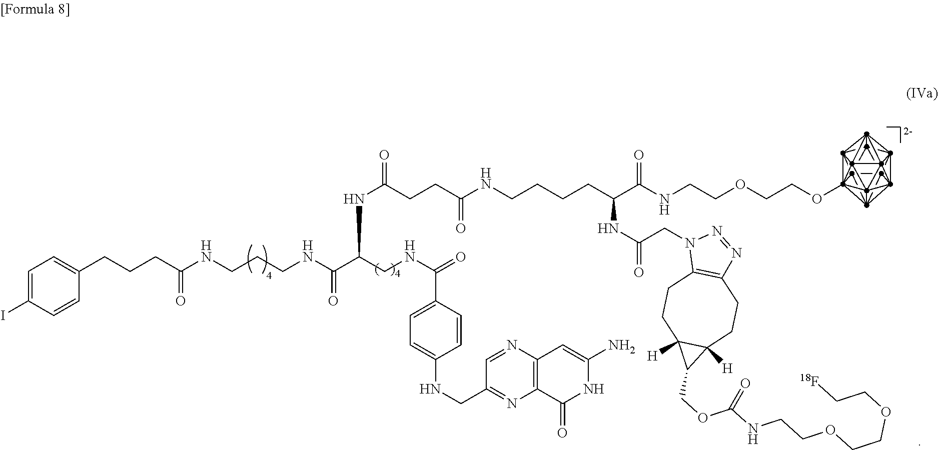

The present specification includes the contents described in the specifications and/or drawings of the Japanese patent applications, Japanese Patent Application No. 2021-039885 and Japanese Patent Application No. 2021-096432, which are the basis of the priority of the present application.

### Advantageous Effects of Invention

The present invention provides a novel boron agent used in BNCT and a novel imaging agent for PET for predicting an effect of the boron agent.

### BRIEF DESCRIPTION OF DRAWINGS

FIG. 1 shows the folate receptor (FRα) expression level in each cancer cell.

FIG. 2 shows results of quantification of the amount of the folate receptor (FRα) by flow cytometry.

FIG. 3 shows a thermal neutron dose-dependent BNCT antitumor effect. (A) Human brain tumor cell U87MG (FRα(+)), (B) human lung cancer cell A549 (FRα(−)). The cells were cultured for 3 hours with an agent ¹⁰B at a concentration of 25 ppm, followed by removal of the agent and irradiated.

FIG. 4 shows (A) pharmacokinetics (7.5 mg (¹⁰B)/kg) and (B) antitumor effects and (C) changes in body weight of a PBC-IP albumin complex and BPA in mice having the human brain tumor cell U87MG (FRα(+)) subcutaneously implanted therein.

FIG. 5 shows the ability of PBC-IP (A) and BPA (B) to selectively accumulate in various cells.

FIG. 6 shows distributions of the boron agent in F98 and C6 orthotopically implanted brain tumor rats.

FIG. 7 shows the BNCT therapeutic effect of PBC-IP on malignant glioblastoma cell (F98 and C6)-implanted rat brain tumor models. Three months after irradiation, 50% survival was confirmed in the PBC-IP administration group, and 70% survival was confirmed in combination with BPA (FIG. 7(A)).

FIG. 8 shows brain tissue staining images of a rat 100 days after BNCT administration (A) and an untreated rat (B).

### DESCRIPTION OF EMBODIMENTS

Hereinafter, the present invention will be described in detail.

### (1) Boron Agent for BNCT

The boron agent for BNCT according to the present invention contains a compound represented by the following formula (I) or (II).

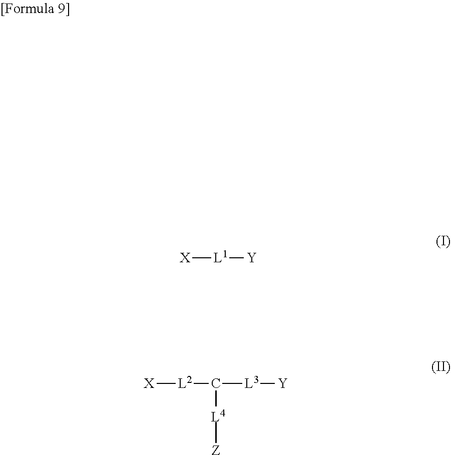

In BNCT (boron neutron capture therapy), a neutron beam is irradiated to a patient or the like who has been given a boron agent in advance, and the reaction between the boron agent and neutrons generates lithium and an a ray in the microenvironment within a single cell to destroy a cancer cell. The “boron agent for BNCT” in the present invention means the boron agent used in this BNCT.

The group X in formulas (I) and (II) is not particularly limited as long as it is a group that binds to albumin, and is preferably a group that non-covalently binds to albumin. This is because if the boron agent is covalently bound to albumin, the boron agent may not be released within the tumor and may be unable to exhibit sufficient efficacy. A large number of groups that bind to albumin and a large number of groups that non-covalently bind to albumin are known, and in the present invention, a suitable group can be appropriately selected among such known groups and used. Specific examples of the group that non-covalently binds to albumin include an iodobutyrate group represented by the following formula (A), a group contained in Evans blue represented by the following formula (B), and a group represented by the following formula (C).

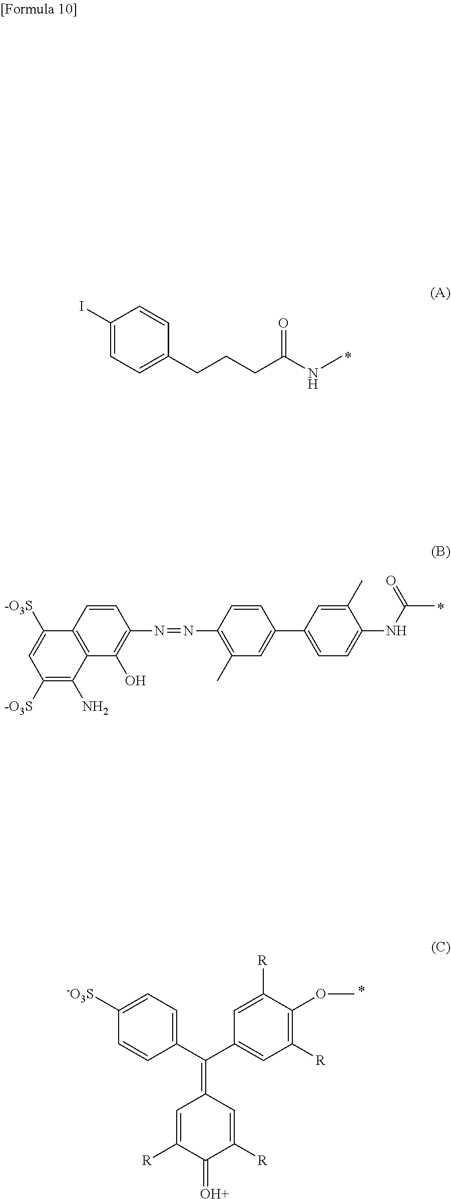

The group Y in formulas (I) and (II) is not particularly limited as long as it is a group containing 1°B, and may be a group derived from a compound having one boron atom in the molecule such as BPA, and is preferably a group derived from a boron cluster. The boron cluster may be any having a polyhedral structure that can be used in boron neutron capture therapy, and examples thereof include closododecaborate ([B₁₂H₁₂]²⁻), ionic closocarborane ([CB₁₁H₁₂]⁻), fat-soluble closocarborane ([C₂B₁₀H₁₂]), nidocarborane ([C₂B₉H₁₁]⁻), a bisdicarbollide metal complex ([(C₂B₉H₁₁)₂M]), and GB10 ([B₁₀H₁₂]²⁻). The boron cluster is preferably a water-soluble boron cluster such as closododecaborate, ionic closocarborane, nidocarborane, or GB10. All of the boron atoms included in the boron cluster may be ¹⁰B, or only a part thereof may be ¹⁰B. Herein, the expression “group derived” as in the phrase “a group derived from a boron cluster” is used, which means, for example, a group derived by removing one hydrogen atom in a boron cluster.

The group Z in formula (II) is not particularly limited as long as it is a group that binds to a folate receptor. A large number of groups that bind to a folate receptor are known, and in the present invention, a suitable group can be appropriately selected among such known groups and used. Specific examples of the group that binds to a folate receptor include a group contained in folic acid represented by the following formula (D).

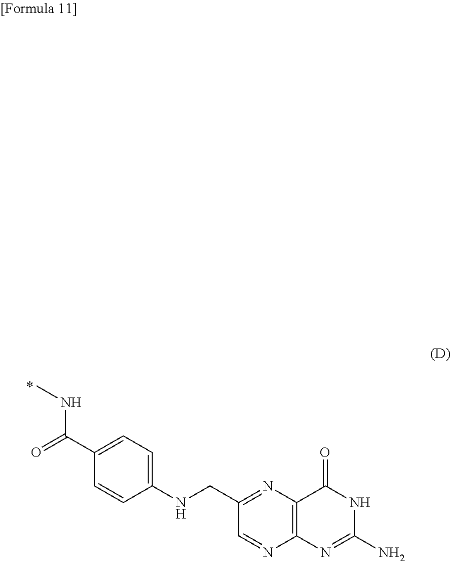 [SMILES: CNC(=O)c1ccc(NCc2cnc3nc(N)[nH]c(=O)c3n2)cc1]

By using a compound that has a group that binds to a folate receptor as a boron agent, the boron agent can be taken up by cancer cells via the folate receptors that are highly expressed in many cancer cells. In addition, BPA, a known boron agent, is taken up by a cell via an amino acid transporter called LAT-1, and the expression of this LAT-1 is low in a BPA-insensitive cancer. A compound having a group that binds to a folate receptor is taken up by a cancer cell not via LAT-1, and thus is also effective against a BPA-insensitive cancer.

The group that binds to a folate receptor is for uptake into a cancer cell, and as shown in Example 6, having such a group improves the binding to albumin. As a result, the retention in blood is improved, and the delivery to cancer cells is also improved.

The groups L¹, L², L³, and L⁴ in formulas (I) and (II) are not particularly limited as long as these are each a divalent group that functions as a spacer, and are each preferably a linear divalent group, and more preferably an alkylene group. However, one or more —CH₂—in the alkylene group may optionally be substituted with —O—, —S—, —NH—, or —CO—. The number of carbon atoms in the alkylene group is not particularly limited as long as it is a number that can sufficiently secure the distance between the two groups, a group that binds to albumin and a group containing ¹⁰B, or the distance among the three groups, a group that binds to albumin, a group containing ¹⁰B, and a group that binds to a folate receptor; and the number is preferably 5 to 20 and more preferably 10 to 15 for L¹, preferably 3 to 15 and more preferably 5 to 10 for L², preferably 5 to 20 and more preferably 10 to 15 for L³, and preferably 2 to 10 and more preferably 3 to 8 for L⁴. Even when —CH₂— in the alkylene group is substituted with —O—, —S—, or —NH—, the resulting group is assumed to have one carbon atom, and this carbon atom is included in the above “number of carbon atoms in the alkylene group.” Specific examples of L¹ include —CH₂—CH₂—CH₂—CH₂—CH₂—CH₂—NH—CH₂—CH₂—O—CH₂—CH₂—O— contained in the compound represented by formula (Ia) and a divalent group having a length identical thereto. Specific examples of L² include-CH₂—CH₂—CH₂—CH₂—CH₂—CH₂—NH—CO-contained in the compound represented by formula (IIa) and a divalent group having a length identical thereto, specific examples of L³ include-NH—CO—CH₂—CH₂—CO—NH—CH₂—CH₂—O—CH₂—CH₂—O-contained in the compound represented by formula (IIa) and a divalent group having a length identical thereto, and specific examples of L⁴ include-CH₂—CH₂—CH₂—CH₂-contained in the compound represented by formula (IIa) and a divalent group having a length identical thereto.

The compounds represented by formula (I) or (II) can be synthesized according to the methods described in the Examples, or according to methods obtained by appropriately altering or modifying those methods with reference to the descriptions thereof. For example, by combining a compound having a group that binds to albumin and a compound having a group containing ¹⁰B, the compound represented by formula (I) can be synthesized, and by combining a compound having a group that binds to albumin and a compound having a group containing ¹⁰B and then combining a compound having a group that binds to a folate receptor, the compound represented by formula (II) can be synthesized.

Specific examples of the compound represented by formula (I) and the compound represented by formula (II) include compounds represented by the following formulas (Ia) and (IIa), respectively.

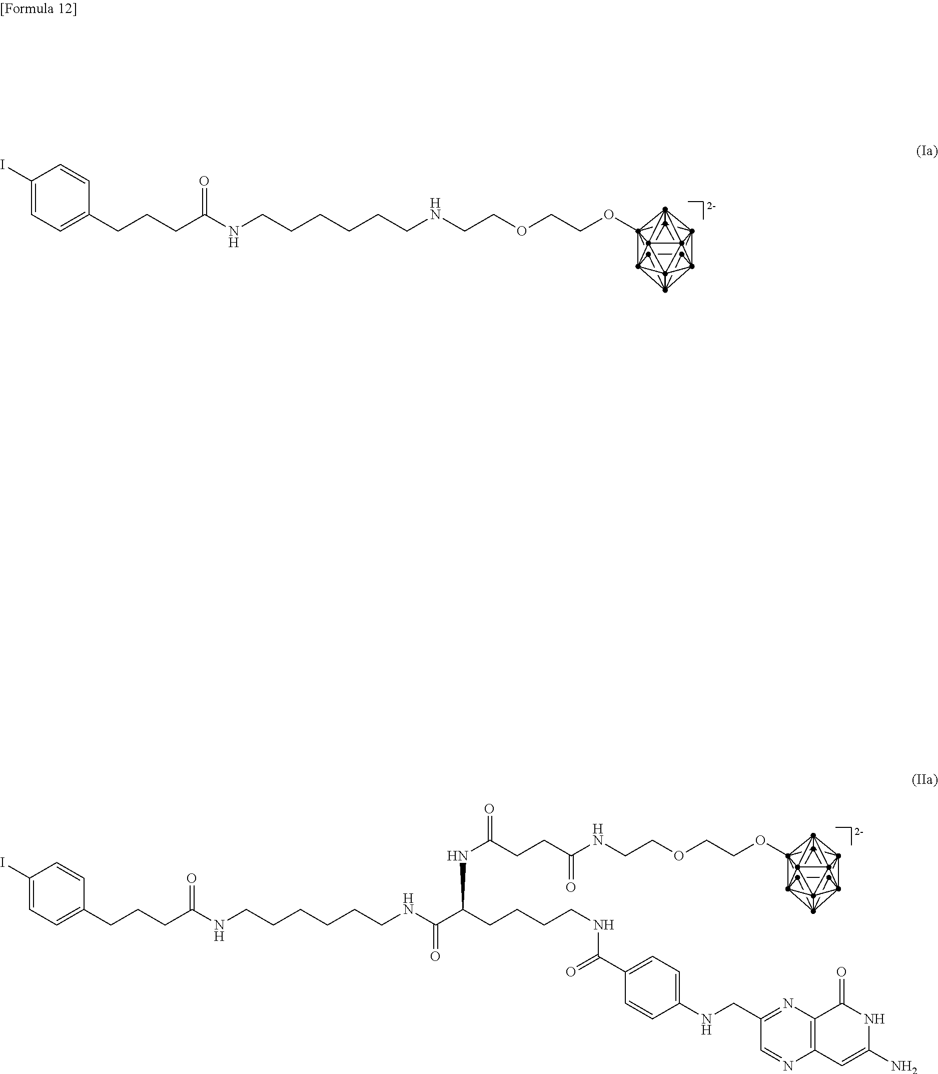

Hereinafter, the compound represented by formula (Ia) may be referred to as “BC-IP,” and the compound represented by formula (IIa) may be referred to as “PBC-IP.”

The boron agent for BNCT according to the present invention is administered to humans or non-human animals. Examples of the non-human animals include mice, rats, hamsters, rabbits, cats, dogs, cows sheep, and monkeys.

Examples of a disease to be treated include, but are not limited to, malignant tumors, such as brain tumor, malignant melanoma, head and neck cancer, lung cancer, liver cancer, thyroid cancer, skin cancer, bladder cancer, mesothelioma, pancreatic cancer, breast cancer, meningioma, and sarcoma.

A patient to be treated is not particularly limited, and is preferably a BPA-insensitive patient. As used herein, the term “BPA-insensitive patient” refers to a patient having a small amount of BPA accumulated in tumor cells. Before administering boron neutron capture therapy with BPA, PET diagnosis using ¹⁸F-BPA is usually made to estimate the amount of BPA accumulated in tumor cells, and boron neutron capture therapy is considered desirable for patients found to have tumor/normal tissue ratio and tumor/blood concentration ratio of 2.5 (or 3) or more in the PET diagnosis. Therefore, in the present invention, patients in whom either one or both of the tumor/normal tissue ratio and the tumor/blood concentration ratio is/are less than 3 or less than 2.5 in the above PET diagnosis can be referred to as “BPA-insensitive patients.”

The boron agent for BNCT according to the present invention can be formulated by mixing the same with pharmaceutically acceptable carriers or diluents according to a known method. The dosage form is not particularly limited, and can be an injection, a tablet, a powder, a granule, a capsule, a liquid, a suppository, a sustained-release preparation, or the like. The administration method is not particularly limited, either, and the agent can be administered orally or parenterally. Examples of the parenteral administration method include an administration method by intradermal, intraperitoneal, intravenous, arterial, or spinal fluid injection, drip infusion, or the like. In addition, local administration by CED (Convection Enhanced Delivery) can also be carried out. The dosage varies depending on the subject of administration, the administration method, or the like, and for example, when the compound represented by formula (I) or (II) is administered to an adult as an injection, the compound represented by formula (I) or (II) can be administered in one to several divided doses per treatment to achieve 5 to 1000 mg/kg of the compound represented by formula (I) and (II) per dose. In addition, before administering the boron agent for BNCT according to the present invention, the imaging agent for PET according to the present invention described later is administered, and based on the biodistribution of the compound represented by formula (III) or (IV) and changes over time thereof, the dosage may be determined.

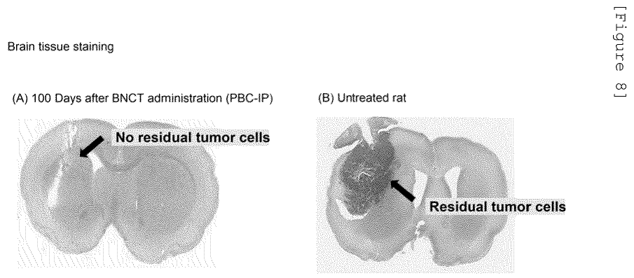

The boron agent for BNCT according to the present invention may be used in combination with a known boron agent for BNCT (for example, BPA or BSH). As shown in FIG. 7(A), when the boron agent for BNCT (PBC-IP) according to the present invention is used in combination with BPA, the survival time prolonging effect is further improved, and thus BPA is preferable as a known boron agent for BNCT to be used in combination.

The boron agent for BNCT according to the present invention can bind to albumin, and thus can also be bound to albumin before administration and administered as an albumin complex.

### (2) Imaging Agent for PET

The imaging agent for PET according to the present invention contains a compound represented by the following formula (III) or (IV).

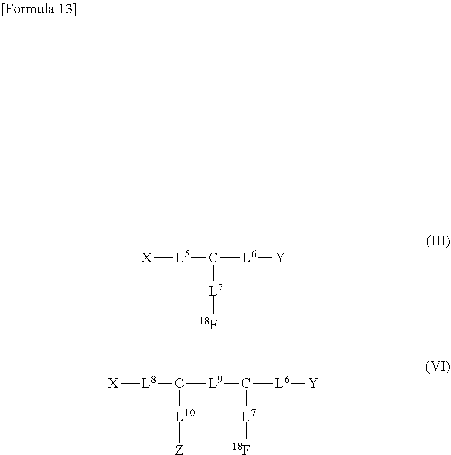

The compounds represented by formulas (III) and (IV) are labeled with ¹⁸F, and thus can emit positrons. The emitted positrons immediately bind to electrons to emit a y ray. By measuring this y ray with an apparatus used for PET, the biodistributions of the compounds represented by formulas (III) and (IV) can be imaged quantitatively and over time. The compounds represented by formulas (III) and (IV) are similar to the compounds represented by formulas (I) and (II), respectively, except that the former is labeled with ¹⁸F, and thus from the biodistributions of the compounds represented by formulas (III) and (IV) and changes over time therein, it is possible to estimate the biodistributions of the compounds represented by formulas (I) and (II) and changes over time therein. Therefore, by administering the compound represented by formula (III) or (IV) to a patient, it is possible to estimate, for example, whether or not the patient is sensitive to the compounds represented by formulas (I) and (II), or how much the compounds represented by (I) and (II) should be administered to obtain an anticancer effect.

Examples of X, Y, and Z in formulas (III) and (IV) include the same groups as those of X, Y, and Z in formulas (I) and (II).

The groups L⁵, L⁶, L⁷, L⁸, L⁹, and L¹⁰ in formulas (III) and (IV) are not particularly limited as long as these are each a divalent group that functions as a spacer, and are each preferably a linear divalent group, and more preferably an alkylene group. However, in L⁵, L⁶, L⁸, L⁹, and L¹⁰, one or more —CH₂— in the alkylene group may optionally be substituted with —O—, —S—, —NH—, or —CO—. In addition, in L⁷, one or more —CH₂— in the alkylene group may optionally be substituted with —O—, —S—, —NH—, or —CO—, and one —CH₂— in the alkylene group may optionally be substituted with a divalent group formed by a click reaction. Here, the divalent group formed by a click reaction is, for example, a divalent group formed by reacting an alkyne and an azide group. A large number of divalent groups formed by a click reaction and a large number of divalent groups formed by reacting an alkyne and an azide group are known, and in the present invention, such a known group can be appropriately selected and used. Examples of the divalent group formed by reacting an alkyne and an azide group include a divalent group formed by reacting bicyclononyne contained in the compound represented by formula (IVa) and an azide group. The number of carbon atoms in the alkylene group is not particularly limited as long as it is a number that can sufficiently secure the distance among the three groups, a group that binds to albumin, a group containing ¹⁰B, and ¹⁸F, or the distance among the four groups, a group that binds to albumin, a group containing ¹⁰B, ¹⁸F, and a group that binds to a folate receptor; and the number is preferably 5 to 25 and more preferably 10 to 20 for L⁵, preferably 3 to 15 and more preferably 5 to 10 for L⁶, preferably 5 to 25 and more preferably 10 to 20 for L⁷, preferably 3 to 15 and more preferably 5 to 10 for L⁸, preferably 3 to 20 and more preferably 5 to 15 for L⁹, and preferably 2 to 10 and more preferably 3 to 8 for L¹⁰. Even when-CH₂-in the alkylene group is substituted with —O—, —S—, or —NH—, the resulting group is assumed to have one carbon atom, and this carbon atom is included in the above “number of carbon atoms in the alkylene group.” On the other hand, when one —CH₂— in the alkylene group in L⁷ is substituted with a divalent group formed by a click reaction, this divalent group is not included in the above “number of carbon atoms in the alkylene group.” Specific examples of L⁶ include —CO—NH—CH₂—CH₂—O—CH₂—CH₂—O— contained in the compound represented by formula (IVa) and a divalent group having a length identical thereto, specific examples of L⁷ include —NH—CO—CH₂-(divalent group formed by click reaction)-CH₂—O—CO—NH—CH₂—CH₂—O—CH₂—CH₂—O—CH₂—CH₂-contained in the compound represented by formula (IVa) and a divalent group having a length identical thereto, specific examples of L⁸ include —CH₂—CH₂—CH₂—CH₂—CH₂—CH₂—NH—CO— contained in the compound represented by formula (IVa) and a divalent group having a length identical thereto, specific examples of L⁹ include —NH—CO—CH₂—CH₂—CO—NH—CH₂—CH₂—CH₂—CH₂-contained in the compound represented by formula (IVa) and a divalent group having a length identical thereto, and specific examples of L¹⁰ include —CH₂—CH₂—CH₂—CH₂-contained in the compound represented by formula (IVa) and a divalent group having a length identical thereto.

The compounds represented by formula (III) or (II) can be synthesized according to the methods described in the Examples, or according to methods obtained by appropriately altering or modifying those methods with reference to the descriptions thereof.

Specific examples of the compound represented by formula (IV) include a compound represented by the following formula (IVa).

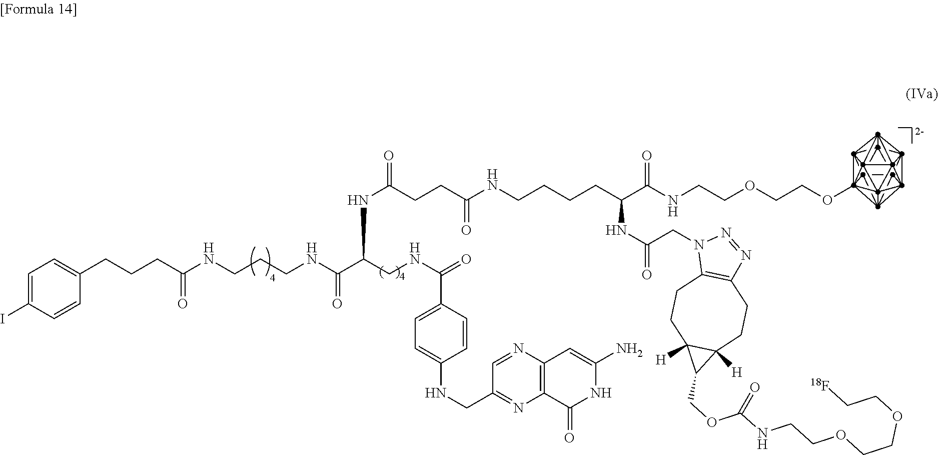

The imaging agent for PET according to the present invention can be formulated by mixing the same with pharmaceutically acceptable carrier or diluent according to a known methods. The dosage form is not particularly limited, and can be an injection, a tablet, a powder, a granule, a capsule, a liquid, a suppository, a sustained-release preparation, or the like. The administration method is not particularly limited, either, and the agent can be administered orally or parenterally (administration by intradermal, intraperitoneal, intravenous, arterial, or spinal fluid injection, drip infusion, or the like). The dosage varies depending on the subject of administration, the administration method, or the like, and for example, when a compound represented by formula (III) or (IV) is administered to an adult as an injection, the compound represented by formula (III) or (IV) can be administered to achieve 1 μg to 1000 μg/kg thereof.

### EXAMPLES

Hereinafter, the present invention will be described in more detail with reference to Examples, but the present invention is not limited to these Examples.

### [Example 1] Synthesis Scheme of PBC-IP

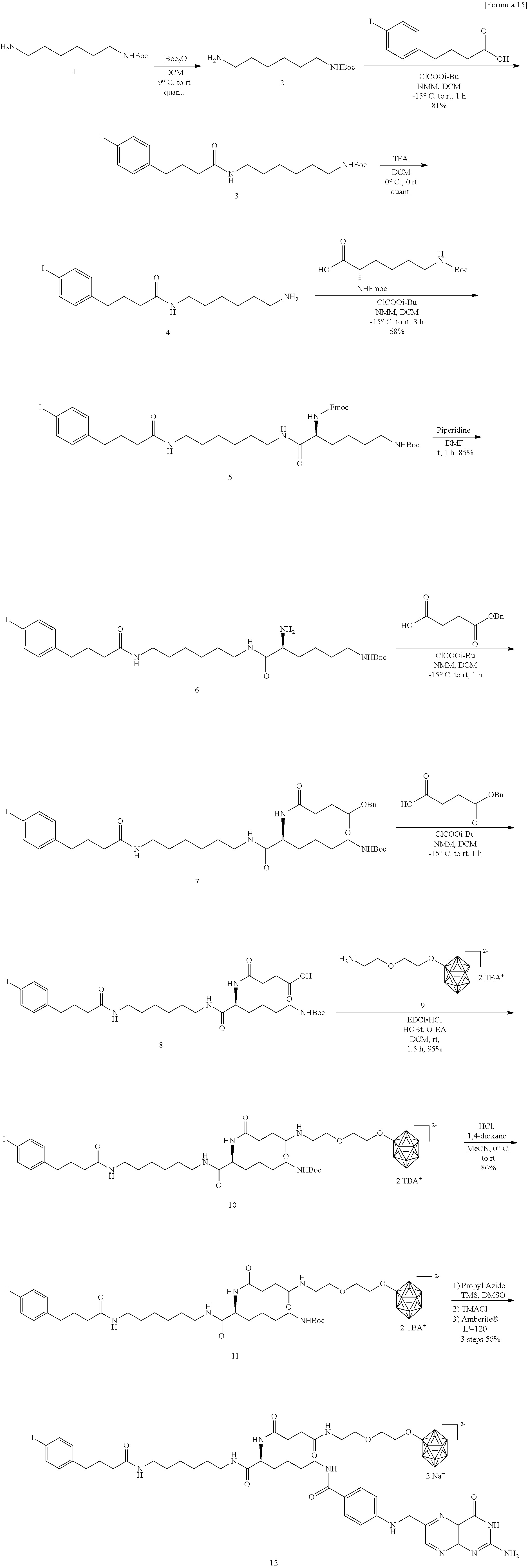

Compound 2 (Li, Y. X.; Qiu, Z. Asian, J. Chem. 2014, 26, 3219), monobenzyl succinate (Isomura, S.; Wirs, ing, P.; Janda, K. D. J. Org. Chem. 2001, 66, 4115), compound 9 (Ishii, S.; Nakamura, H. J. Organomet. Chem. 2018, 865, 178), pteroyl azide (Luo, J.; Smith, M. D.; Lantrip, D. A.; Wang, S.; Fuchs, P. L. J. Am. Chem. Soc. 1997, 119, 10004) were synthesized according to known methods.

### Synthesis of Compound 3 from Compound 2

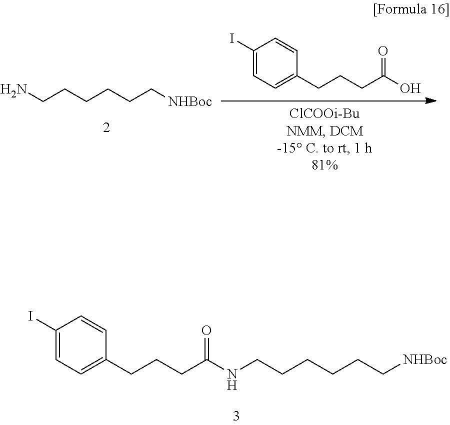 [SMILES: O=C(O)CCCc1ccc(I)cc1 ; CC(C)(C)OC(=O)NCCCCCCN ; CC(C)(C)OC(=O)NCCCCCCNC(=O)CCCc1ccc(I)cc1]

4-Iodophenylbutyric acid (4.787 g, 16.5 mmol) and N-methylmorpholine (1.91 mL, 17.3 mmol) were dissolved in dichloromethane (33 mL) in an argon atmosphere, and then isobutyl chloroformate (2.28 mL, 17.3 mmol) was added dropwise at −15° C. The resulting mixture was stirred at −15° C. for 20 minutes, and then a dichloromethane solution (33 mL) of compound 2 (4.1049 g, 19.0 mmol) and N-methylmorpholine (1.81 mL, 16.5 mmol) was added dropwise at −15° C. After dropwise addition, the resulting mixture was stirred at room temperature for 30 minutes, then water was added to stop the reaction, and the mixture was extracted three times with dichloromethane. The organic layer was dried over magnesium sulfate and filtered, and then the solvent was removed under reduced pressure. The resulting crude product was dissolved in chloroform/methanol at 60° C., hexane was added, and then the resulting mixture was cooled to room temperature. The resulting solid was filtered and dried under reduced pressure to obtain compound 3 (6.5081 g, yield of 81%) as a white solid.

¹H NMR (400 MHZ, DMSO-d₆) δ 7.71 (t, J=5.4 Hz, 1H), 7.62 (d, J=8.2 Hz, 2H), 7.00 (d, J=8.2 Hz, 2H), 6.74 (brs, 1H), 3.00 (q, J=6.0 Hz, 2H), 2.88 (q, J=6.4 Hz, 2H), 2.50 (t, J=7.3 Hz, 2H), 2.03 (t, J=7.3 Hz, 2H), 1.75 (quint, J=7.5 Hz, 2H), 1.36 (brs, 13H), 1.23-1.21 (m, 4H).

### Synthesis of Compound 4 from Compound 3

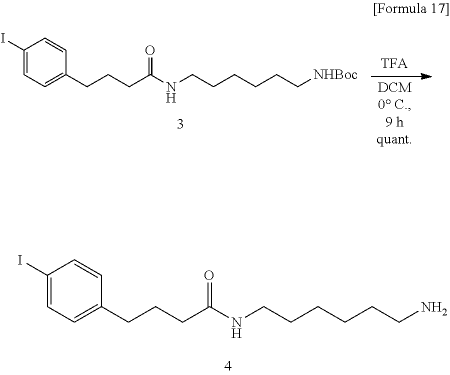 [SMILES: CC(C)(C)OC(=O)NCCCCCCNC(=O)CCCc1ccc(I)cc1 ; NCCCCCCNC(=O)CCCc1ccc(I)cc1]

Compound 3 (6.300 g, 12.9 mmol) was dissolved in dichloromethane (65 mL) in an argon atmosphere, and then trifluoroacetic acid (8.0 mL) was added dropwise at 0° C. The resulting mixture was stirred at 0° C. for 9 hours, then a 1 M sodium hydroxide aqueous solution was added to stop the reaction, and the mixture was extracted three times with dichloromethane. The organic layer was dried over magnesium sulfate and filtered, and then the solvent was removed under reduced pressure to quantitatively obtain compound 4 (5.446 g) as a white solid. ¹H NMR (400 MHZ, DMSO-d₆) δ 7.73 (d, J=5.3 Hz, 1H), 8 7.78-7.66 (m, 2H), 7.63 (d, J=8.2 Hz, 1H), 7.01 (d, J=8.3 Hz, 2H), 3.01 (q, J=6.8 Hz, 3H), 2.61 (qt, J=7.0 Hz, 3H), 2.04 (t, J=7.4 Hz, 1H), 1.76 (quin, J=7.6 Hz, 2H), 1.48-1.32 (m, 4H), 1.31-1.18 (m, 4H).

### Synthesis of Compound 5 from Compound 4

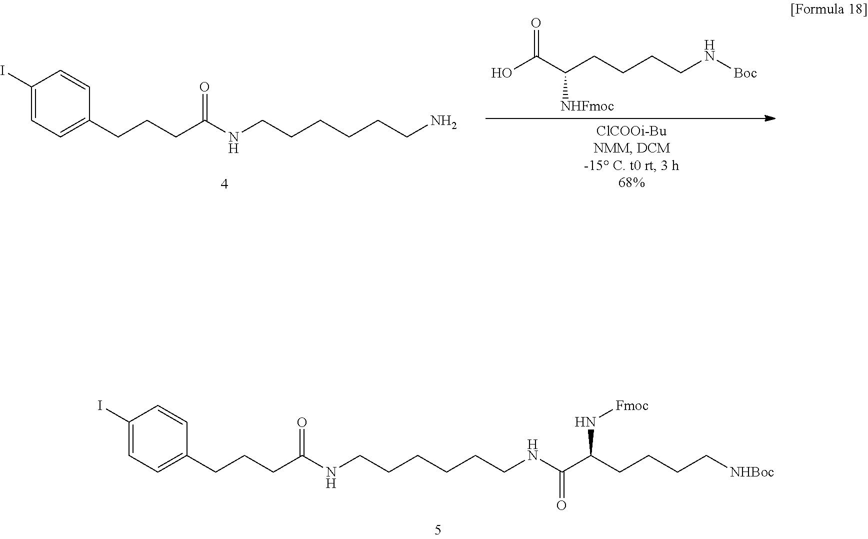

Fmoc-Lys (Boc)-OH (6.150 g, 13.1 mmol) and N-methylmorpholine (1.44 mL, 13.1 mmol) were dissolved in dichloromethane (40 mL) in an argon atmosphere, and then isobutyl chloroformate (1.72 mL, 13.1 mmol) was added dropwise at −15° C. The resulting mixture was stirred at −15° C. for 20 minutes, and then a dichloromethane solution (40 mL) of compound 4 (4.854 g, 12.5 mmol) and N-methylmorpholine (1.30 mL, 12.5 mmol) was added dropwise at −15° C. After dropwise addition, the resulting mixture was stirred at room temperature for 60 minutes, then water was added to stop the reaction, and the mixture was extracted three times with dichloromethane. The organic layer was washed with saturated brine, dried over magnesium sulfate, and filtered, and then the solvent was removed under reduced pressure. The resulting crude product was dissolved in chloroform/methanol at 60° C., hexane was added, and then the resulting mixture was cooled to room temperature. The resulting solid was filtered and dried under reduced pressure to obtain compound 5 (7.1211 g, yield of 68%) as a white solid. ¹H NMR (500 MHZ, DMSO-d₆) δ 7.89 (d, J=7.5 Hz, 2H), 8 7.82 (t, J=5.3 Hz, 1H), 8 7.78-7.66 (m, 2H), 7.62 (d, J=8.1 Hz, 1H), 7. 46-7. 37 (m, 3H), 7.33 (t, J=7.3 Hz, 2H), 7.00 (d, J=8.2 Hz, 2H), 6.77 (d, J=5.1 Hz, 1H), 4.35-4.20 (m, 3H), 3.98-3.88 (m, 1H), 3.13-2.96 (m, 4H), 2.89 (t, J=5.0 Hz, 1H), 2.50-2.45 (m, 2H), 2.04 (t, J=7.4 Hz, 1H), 1.76 (quin, J=7.5 Hz, 2H), 1.65-1.47 (m, 2H), 1.44-1.32 (m, 15H), 1.31-1.17 (m, 6H).

### Synthesis of Compound 6 from Compound 5

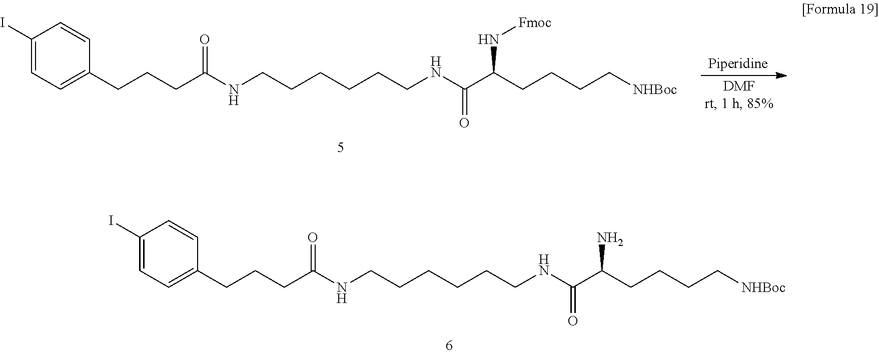

Compound 5 (6.513 g, 7.76 mmol) was dissolved in N, N-dimethylformamide (24 mL) in an argon atmosphere, and then piperidine (2.4 mL) was added at room temperature. The resulting mixture was stirred for 60 minutes, then water was added to stop the reaction, and the mixture was extracted three times with dichloromethane. The organic layer was washed with 1 M hydrochloric acid and a saturated sodium hydrogen carbonate aqueous solution, dried over magnesium sulfate, and filtered, and then the solvent was removed under reduced pressure. The resulting crude product was purified by silica gel column chromatography (dichloromethane/methanol=90:10) to obtain compound 6 (4.078 g, yield of 85%) as a white solid.

¹H NMR (500 MHZ, DMSO-d₆) δ 7.89 (d, J=7.5 Hz, 2H), δ 7.82 (t, J=5.3 Hz, 1H), δ 7.78-7.66 (m, 2H), 7.62 (d, J=8.1 Hz, 1H), 7.46-7.37 (m, 3H), 7.33 (t, J=7.3 Hz, 2H), 7.00 (d, J=8.2 Hz, 2H), 6.77 (d, J=5.1 Hz, 1H), 4.35-4.20 (m, 3H), 3.98-3.88 (m, 1H), 3.13-2.96 (m, 4H), 2.89 (t, J=5.0 Hz, 1H), 2.50-2.45 (m, 2H), 2.04 (t, J=7.4 Hz, 1H), 1.76 (quin, J=7.5 Hz, 2H), 1.65-1.47 (m, 2H), 1.44-1.32 (m, 15H), 1.31-1.17 (m, 6H).

### Synthesis of Compound 8 from Compound 6

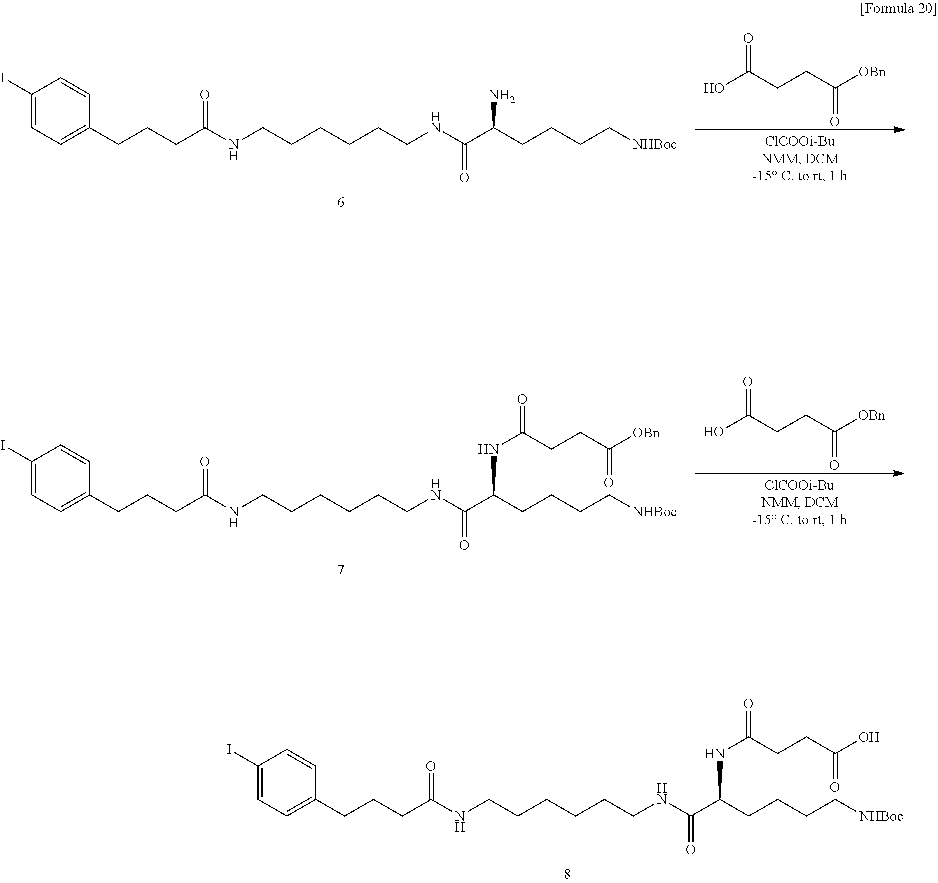 [SMILES: CC(C)(C)OC(=O)NCCCC[C@H](N)C(=O)NCCCCCCNC(=O)CCCc1ccc(I)cc1 ; O=C(O)CCC(=O)OCc1ccccc1 ; CC(C)(C)OC(=O)NCCCC[C@H](NC(=O)CCC(=O)OCc1ccccc1)C(=O)NCCCCCCNC(=O)CCCc1ccc(I)cc1 ; O=C(O)CCC(=O)OCc1ccccc1 ; CC(C)(C)OC(=O)NCCCC[C@H](NC(=O)CCC(=O)O)C(=O)NCCCCCCNC(=O)CCCc1ccc(I)cc1]

Monobenzyl succinate (687.1 mg, 3.3 mmol) and N-methylmorpholine (363 μL, 3.30 mmol) were dissolved in dichloromethane (9.0 mL) in an argon atmosphere, and then isobutyl chloroformate (433 μL, 3.30 mmol) was added dropwise at −15° C. The resulting mixture was stirred at −15° C. for 20 minutes, and then a dichloromethane solution (9.0 mL) of compound 6 (1.850 g, 3.00 mmol) and N-methylmorpholine (330 μL, 3.00 mmol) was added dropwise at −15° C. After dropwise addition, the resulting mixture was stirred at room temperature for 60 minutes, then water was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (dichloromethane/methanol=95:5) to obtain a mixture containing compound 7.

The obtained mixture was dissolved in methanol (6.0 mL) and distilled water (1.5 mL), and then lithium hydroxide monohydrate (378 mg, 9.00 mmol) was added at room temperature. The resulting mixture was stirred for 30 minutes, then 1 M HCl was added to stop the reaction, and the mixture was extracted three times with dichloromethane. The organic layer was washed with saturated brine, dried over magnesium sulfate, and filtered, and then the solvent was removed under reduced pressure. The resulting solid was washed with diethyl ether and dried under reduced pressure to obtain compound 8 (2.120 g, yield of 99% over two steps) as a white solid.

¹H NMR (500 MHZ, DMSO-d₆) δ 7.95 (d, J=8.1 Hz, 1H), 7.78-7.71 (m, 2H), 7.61 (d, J=8.3 Hz, 2H), 7.00 (d, J=8.3 Hz, 2H), 6.72 (t, J=5.5 Hz, 1H), 4.17-4.11 (m, 1H), 3.09-2.94 (m, 4H), 2.94-2.82 (m, 2H), 2.6-2.34 (m, 6H), 2.04 (d, J=7.4 Hz, 2H), 1.76 (quin, J=7.4 Hz, 2H), 1.64-1.41 (m, 2H), 1.41-1.29 (m, 15H), 1.29-1.14 (m, 6H).

### Synthesis of Compound 10 from Compound 8

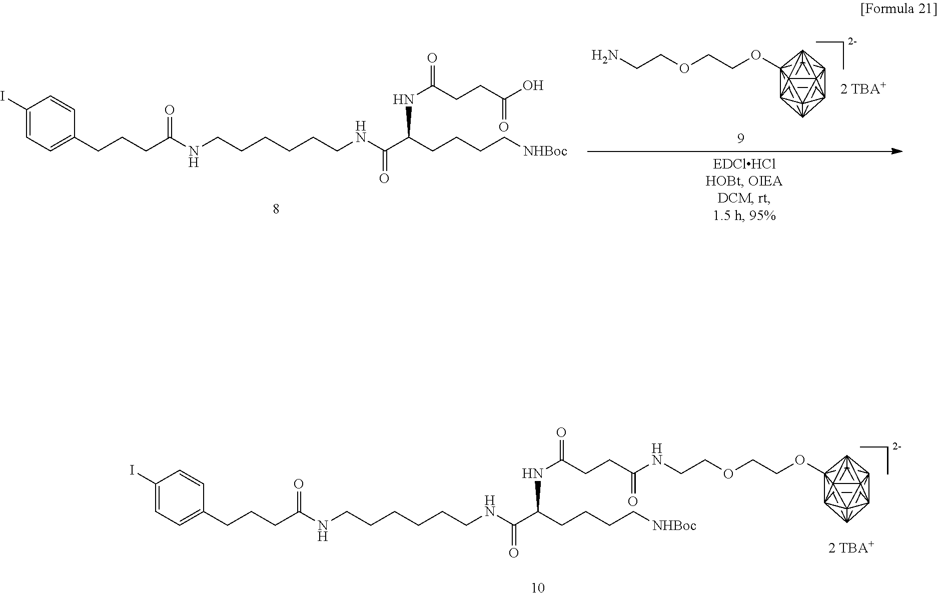

Compound 8 (716.7 mg, 1.00 mmol), compound 9 (730.0 mg, 1.00 mmol), and HOBt·H₂O (168.5 mg, 1.10 mmol) were dissolved in dichloromethane (6.0 mL) in an argon atmosphere, and then diisopropylethylamine (209 μL, 1.20 mmol) and EDCI·HCl (210.9 mg, 1.10 mmol) were added at room temperature. The resulting mixture was stirred for 1.5 hours, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (dichloromethane/methanol=95:5) to obtain compound 10 (1.3631 g, yield of 95%) as a white amorphous solid.

¹H NMR (500 MHZ, DMSO-d₆) δ 8.01 (t, J=5.4 Hz, 1H), 7.96 (d, J=8.0 Hz, 1H), 7.72 (t, J=5.5 Hz, 1H), 7.71 (t, J=8.2 Hz, 1H), 7.62 (d, J=8.3 Hz, 1H), 7.01 (d, J=8.2 Hz, 1H), 6.73 (t, J=5.3 Hz, 1H), 4.10-4.03 (m, 1H), 3.42-3.33 (m, 6H), 3.22-3.10 (m, 18H), 3.04-2.97 (m, 4H), 2.90-2.83 (m, 2H), 2.57-2.32 (m, 4H), 2.04 (t, J=7.5 Hz, 2H), 1.80-1.70 (quin, J=7.5 Hz, 2H), 1.57 (quin, J=7.8 Hz, 16H), 1.41-1.16 (m, 37H), 0.93 (t, J=7.4 Hz, 24H). HRMS (ESI-TOF): calcd for [C₃₅H₆₈B₁₂IN₅O₈]²⁻ 471.7667: found 471.7667.

### Synthesis of Compound 11 from Compound 10

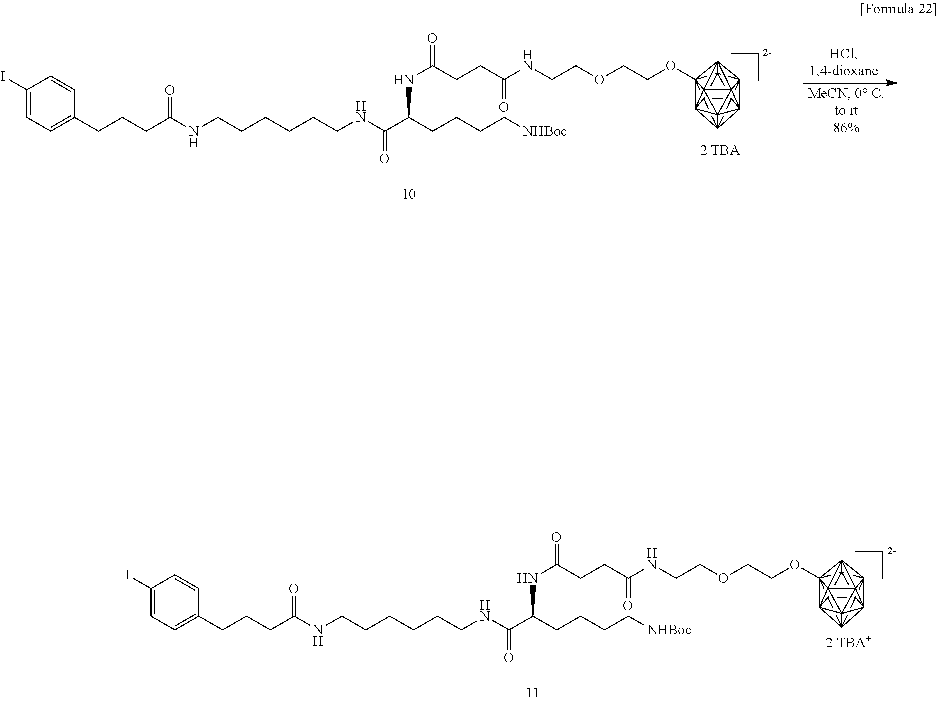

Compound 10 (571.2 mg, 0.400 mmol) was dissolved in acetonitrile (6.0 mL) in an argon atmosphere, and then a 1, 4-dioxane solution (4 M, 2.0 mL) of hydrochloric acid was added at 0° C. The resulting mixture was stirred for 60 minutes at room temperature, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure to obtain compound 11 (453.6 mg, yield of 86%) as a white amorphous solid.

¹H NMR (500 MHZ, DMSO-d₆) δ 8.00 (s, J=5.5 Hz, 1H), 7.95 (d, J=8.1 Hz, 1H), 7.84 (t, J=5.6 Hz, 1H), 7.72 (t, J=5.5 Hz, 1H), 7.62 (d, J=8.3 Hz, 1H), 7.01 (d, J=8.3 Hz, 1H), 4.13-4.04 (m, 1H), 3.42-3.33 (m, 6H), 3.16 (t, J=8.5 Hz, 16H), 3.04-2.93 (m, 4H), 2.45-2.32 (m, 4H), 2.04 (t, J=7.4 Hz, 2H), 1.80-1.70 (m, 2H), 1.57 (quin, J=8.2 Hz, 16H), 1.41-1.16 (m, 28H), 0.93 (t, J=7.5 Hz, 24H). HRMS (ESI-TOF): calcd for [C₃₀H₆₀B₁₂IN₅O₆]²⁻ 421.7403: found 421.7393.

### Synthesis of Compound 12 from Compound 11

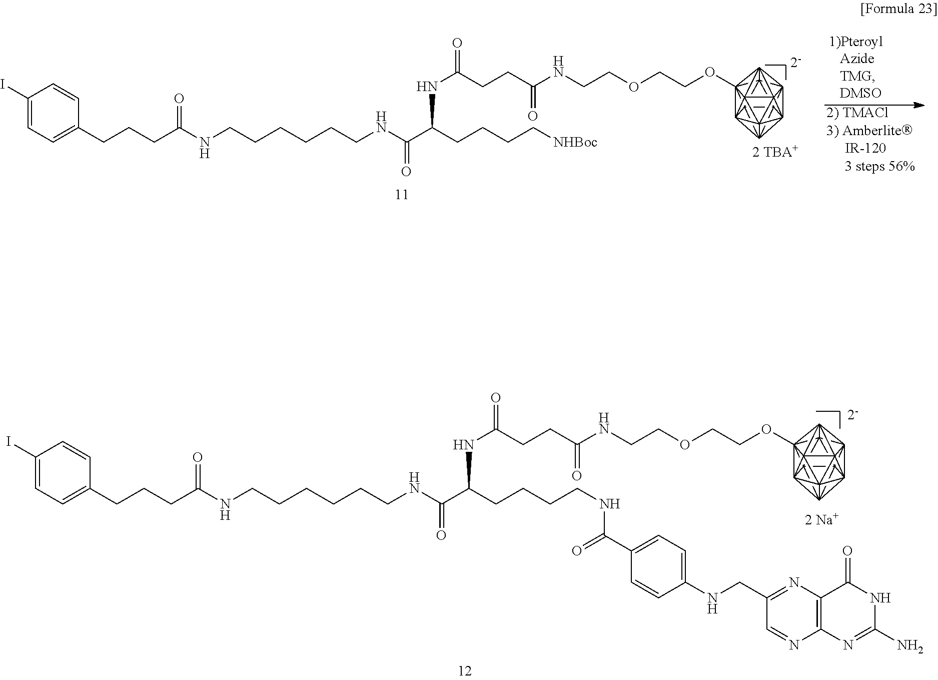

Compound 11 (1.277 g, 0.961 mmol) and pteroyl azide (324.0 mg, 0.961 mmol) were dissolved in dimethylsulfoxide (4.0 mL) in an argon atmosphere, and then 1, 1, 3, 3-tetramethylguanidine (481 UL, 3.84 mmol) was added at room temperature. The resulting mixture was stirred at room temperature for 4 hours, then the mixture was diluted with dichloromethane/methanol, and diethyl ether was added to precipitate a compound. The mixture was filtered, and the resulting yellow solid was washed with diethyl ether and then dried under reduced pressure.

The resulting yellow solid was dissolved in dichloromethane (3.0 mL) and methanol (3.0 mL), and a methanol solution (3.0 mL) of tetramethylammonium chloride (1.053 g, 9.61 mmol) was added at room temperature. The resulting mixture was stirred for 30 minutes, then the mixture was filtered, and the resulting orange solid was washed with ethanol/methanol (1:1, 20 mL) and then dried under reduced pressure.

The resulting orange solid was dissolved in acetonitrile (6.0 mL) and distilled water (6.0 mL), and Amberlite (registered trademark) IR-120 (12.0 g) was added at room temperature. The resulting mixture was stirred for 12 hours and then filtered, the organic solvent was removed under reduced pressure, and further, water was removed by freeze-drying to obtain compound 12 (609.6 mg, 3-step yield of 56%) as an orange solid. ¹H NMR (500 MHZ, DMSO-d₆) δ 8.71 (s, 1H), 8.04-7.78 (m, 7H), 7.84 (s, 1H), 7.71 (d, J=6.2 Hz, 2H), 7.60-7.55 (m, 4H), 7.00 (d, J=8.2 Hz, 2H), 4.54 (s, 2H), 4.12-4.02 (m, 2H), 3.50 (t, J=5.4 Hz, 1H), 3.46-3.30 (m, 4H), 3.23-3.09 (m, 4H), 3.09-2.93 (m, 4H), 2.33 (s, 2H), 2.04 (t, J=7.4 Hz, 2H), 1.81-1.69 (m, 2H), 1.69-1.59 (m, 1H), 1.59-1.16 (m, 12H). HRMS (ESI-TOF): calcd for [C₄₄H₇₀B₁₂IN₁₁O₈]²⁻, 568. 7840: found 568.7834.

### [Example 2] Synthesis of BC-IP

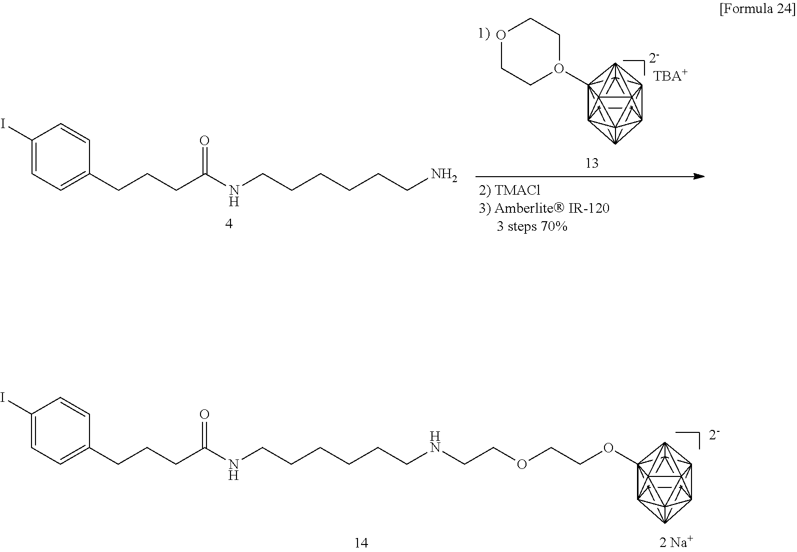

Compound 13 (Ishii, S.; Nakamura, H. J. Organomet. Chem. 2018, 865, 178) was synthesized according to a known method.

### Synthesis of Compound 14 from Compound 13

Compound 4 (448.5 mg, 1.155 mmol) was dissolved in acetonitrile (5.0 mL) in an argon atmosphere, and then potassium carbonate (1.596 g, 11.55 mmol) and compound 13 (619.7 mg, 1.155 mmol) were added at room temperature. The resulting mixture was stirred under heating reflux for 8 hours, then water was added to stop the reaction, and the mixture was extracted three times with dichloromethane. The organic layer was diluted, dried over magnesium sulfate, and filtered, and then the solvent was removed under reduced pressure.

The residue was dissolved in dichloromethane (0.8 mL) and ethanol (6.5 mL), and a methanol solution (6.5 mL) of tetramethylammonium chloride (1.458 g, 13.3 mmol) was added at room temperature. The resulting mixture was stirred for 30 minutes, then the mixture was filtered, and the resulting white solid was washed with ethanol/methanol (1:1, 12 mL) and then dried under reduced pressure.

The resulting white solid was dissolved in acetonitrile (4.0 mL) and distilled water (4.0 mL), and Amberlite (registered trademark) IR-120 (7.0 g) was added at room temperature. The resulting mixture was stirred for 12 hours and then filtered, the organic solvent was removed under reduced pressure, and further, water was removed by freeze-drying to obtain compound 14 (500.4 mg, 3-step yield of 70%) as a white solid.

¹H NMR (400 MHZ, D₂O) δ 7.65 (d, J=8.0 Hz, 2H), 7.13 (d, J=8.0 Hz, 2H), 3.83-3.62 (m, 8H), 3.29-3.23 (m, 2H), 3.16-3.11 (m, 2H), 2.66-2.61 (m, 2H), 2.31-2.24 (m, 2H), 1.96-1.92 (m, 2H), 1.76-1.73 (m, 2H), 1.54-1.49 (m, 2H), 1.44-1.35 (m, 4H).

### [Example 3] Synthesis of Molecule PBC-IP-N3 for PET imaging

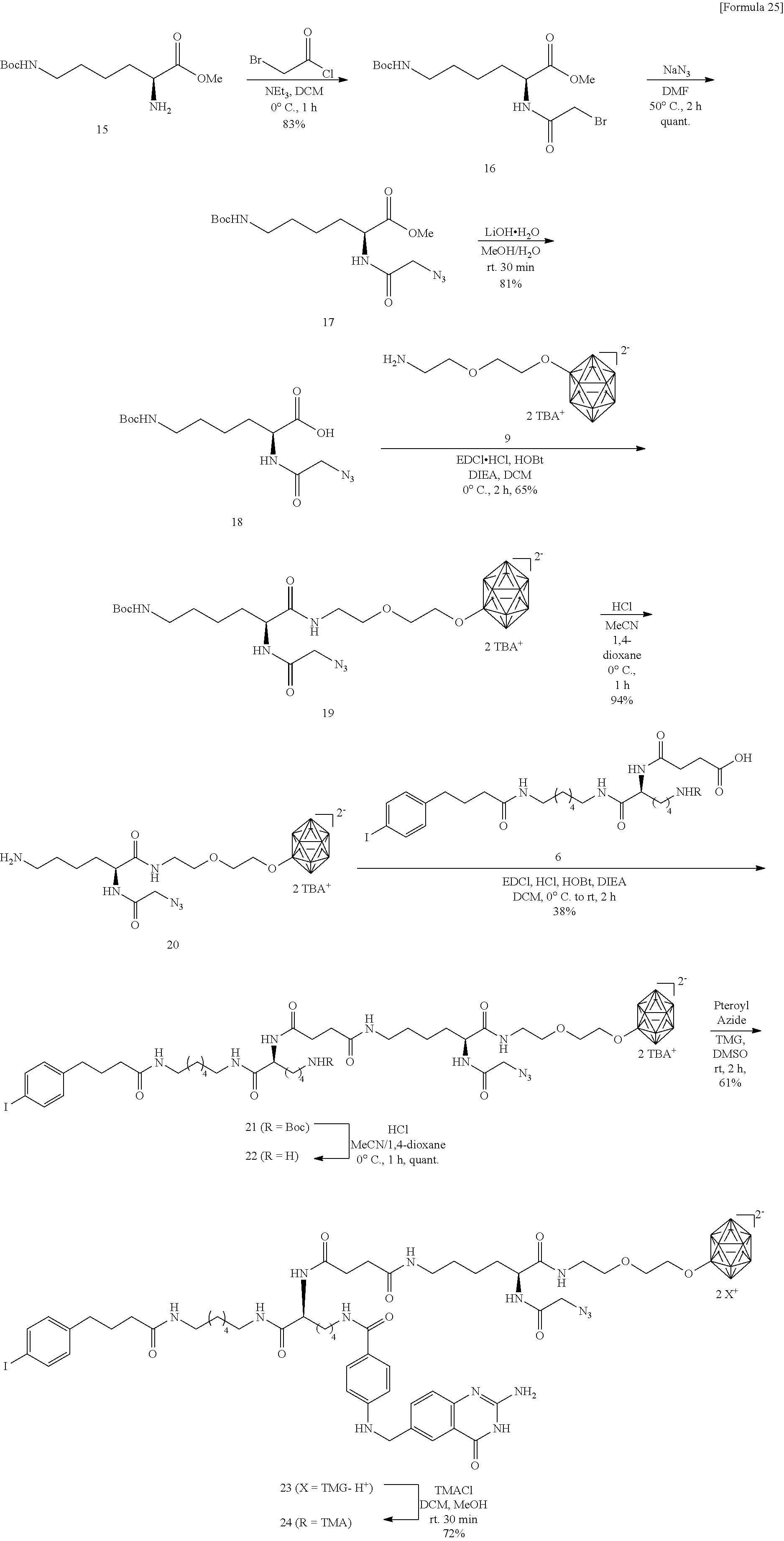

Compound 15 (Robertson, M.; Bremner, J. B.; Coates, J.; Deadman, J.; Keller, P. A.; Pyne, S. G.; Somphol, K.; Rhodes, D. I. Eur. J. Med. Chem. 2011, 46, 4201) was synthesized according to a known method.

### Synthesis of Compound 16 from Compound 15

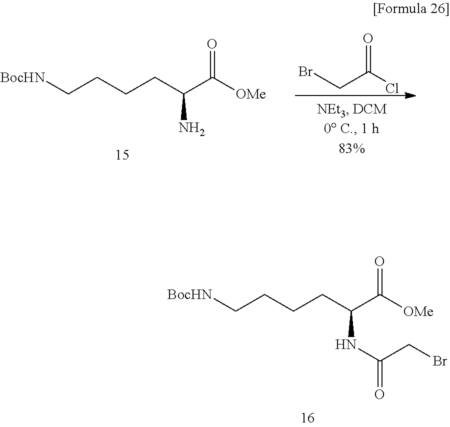 [SMILES: COC(=O)[C@@H](N)CCCCNC(=O)OC(C)(C)C ; O=C(Cl)CBr ; COC(=O)[C@H](CCCCNC(=O)OC(C)(C)C)NC(=O)CBr]

Compound 15 (1.041 g, 4.00 mmol) was dissolved in dichloromethane (12 mL) in an argon atmosphere, and then triethylamine (836 μL, 6.00 mmol) and bromoacetyl chloride (362 μL, 4.40 mmol) were added at 0° C. The resulting mixture was stirred for 50 minutes, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (hexane/ethyl acetate=55:45) to obtain compound 16 (1.258 g, yield of 83%) as a yellow liquid. ¹H NMR (400 MHZ, CDCl₃) δ 6.99 (d, J=1.9 Hz, 1H), 4.60-4.54 (m, 2H), 3.88 (s, 2H), 3.75 (s, 3H), 3.09 (q, J=6.3 Hz, 2H), 1.93-1.84 (m, 1H), 1.78-1.68 (m, 1H), 1.53-1.43 (m, 13H).

### Synthesis of Compound 17 from Compound 16

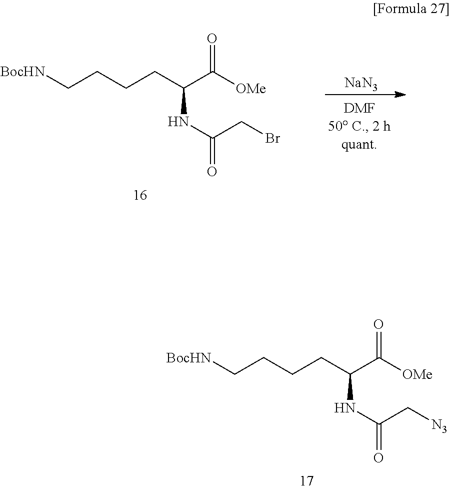 [SMILES: COC(=O)[C@H](CCCCNC(=O)OC(C)(C)C)NC(=O)CBr ; COC(=O)[C@H](CCCCNC(=O)OC(C)(C)C)NC(=O)CN=[N+]=[N-] ; "NaN3"]

Compound 16 (3.014 g, 7.91 mmol) was dissolved in N, N-dimethylformamide (15 mL) in an argon atmosphere, and then sodium azide ((1.541 g, 23.7 mmol) was added at room temperature. The resulting mixture was stirred at 50° C. for 2 hours, then water was added to stop the reaction, and the mixture was extracted three times with ethyl acetate. The organic layer was washed with 1 M hydrochloric acid, dried over magnesium sulfate, and filtered, and then the solvent was removed under reduced pressure. The resulting crude product was purified by silica gel column chromatography (hexane/ethyl acetate=55:45) to quantitatively obtain compound 17 (2.7477 g) as a colorless liquid.

¹H NMR (400 MHZ, CDCl₃) δ 6.81 (d, J=6.8 Hz, 1H), δ 4.62-4.57 (m, 2H), 4.04-3.95 (m, 2H), 3.74 (s, 3H), 3.09 (q, J=6.3 Hz, 2H), 1.91-1.82 (m, 1H), 1.76-1.67 (m, 1H), 1.52-1.42 (m, 11H), 1.37-1.30 (m, 2H).

### Synthesis of Compound 18 from Compound 17

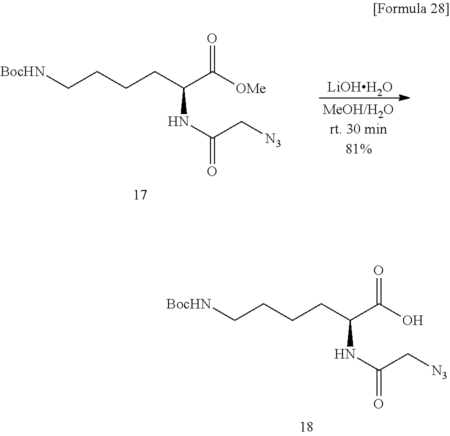 [SMILES: COC(=O)[C@H](CCCCNC(=O)OC(C)(C)C)NC(=O)CN=[N+]=[N-] ; "LiOH·H2O" ; CC(C)(C)OC(=O)NCCCC[C@H](NC(=O)CN=[N+]=[N-])C(=O)O]

Compound 17 (1.889 g, 5.50 mmol) was dissolved in methanol (17.6 mL) and distilled water (4.4 mL), and then lithium hydroxide monohydrate (1.154 g, 27.5 mmol) was added at room temperature. The resulting mixture was stirred for 30 minutes, then 1 M HCl was added to stop the reaction, and the mixture was extracted three times with dichloromethane. The organic layer was washed with saturated brine, dried over magnesium sulfate, and filtered, and then the solvent was removed under reduced pressure to obtain compound 18 (1.4758 g, yield of 81%) as a yellow liquid.

¹H NMR (400 MHZ, CDCl₃) δ 11.4 (brs, 1H), δ 7.17 (d, J=7.8 Hz, 1H), 4.56 (brs, 1H), 3.98 (s, 2H), 3.04 (brs, 2H), 1.92-1.83 (m, 1H), 1.77-1.68 (m, 1H), 1.47-1.35 (m, 13H).

### Synthesis of Compound 19 from Compound 18

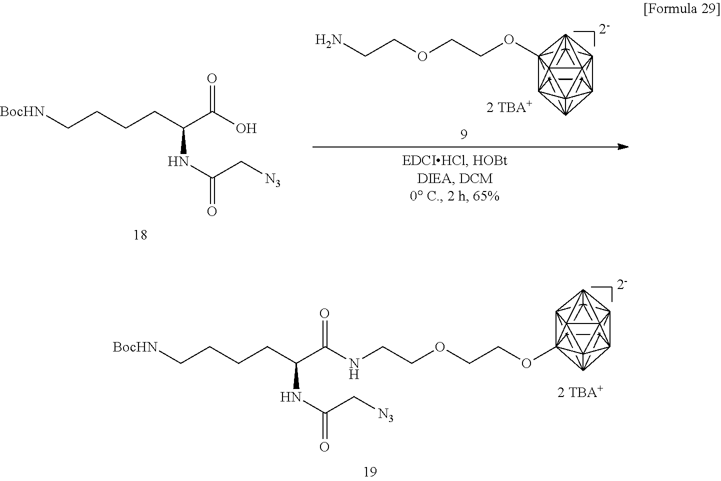

Compound 18 (420.5 mg, 1.28 mmol), compound 9 (931.7 mg, 1.28 mmol), and HOBt·H₂O (183.0 mg, 1.41 mmol) were dissolved in dichloromethane (7.7 mL) in an argon atmosphere, and then diisopropylethylamine (267 μL, 1.53 mmol) and EDCI·HCl (269.5 mg, 1.41 mmol) were added at 0° C. The resulting mixture was stirred at 0° C. for 2 hours, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (dichloromethane/methanol=98:2) to obtain compound 19 (869.7 mg, yield of 65%) as a white amorphous solid.

¹H NMR (400 MHZ, CDCl₃) δ 8.69 (brs, 1H), 7.41 (d, J=9.1 Hz, 1H), 4.77 (brs, 1H), 4.46 (q, J=7.4 Hz, 1H), 4.09 (d, J=16.1 Hz, 1H), 4.01 (d, J=16.1 Hz, 1H), 3.94-3.66 (m, 5H), 3.54-3.51 (m, 1H), 3.41-3.36 (m, 1H), 3.29-3.16 (m, 17H), 3.06 (brs, 2H), 1.95-1.86 (m, 4H), 1.67-1.59 (m, 16H), 1.49-1.39 (m, 27H), 0.99 (t, J=7.3 Hz, 2H); ¹³C NMR (125 MHZ, CDCl₃) δ 172.1, 167.8, 156.0, 78.5, 71.8, 69.9, 69.2, 58.9, 54.3, 51.9, 41.3, 40.3, 32., 29.3, 28.4, 24.1, 23.1, 19.7, 13.7.

### Synthesis of Compound 20 from Compound 19

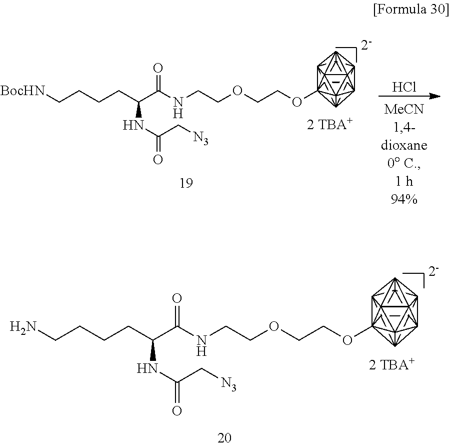

Compound 19 (860.0 mg, 0.826 mmol) was dissolved in acetonitrile (12.0 mL) in an argon atmosphere, and then a 1, 4-dioxane solution (4.0 M, 4.0 mL) of hydrochloric acid was added at 0° C. The resulting mixture was stirred at 0° C. for 1 hour, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure to obtain compound 20 (728.8 mg, yield of 94%) as a white amorphous solid.

HRMS (ESI-TOF): calcd for [C₁₂H₃₄B₁₂N₆O₄]²⁻ 455.3766: found 455.3773.

### Synthesis of Compound 22 from Compound 20

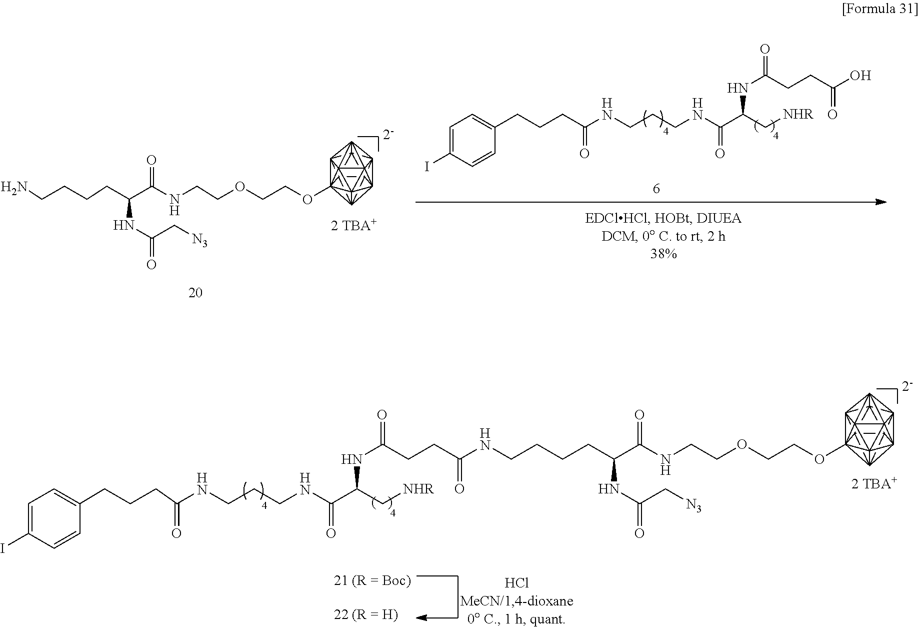

Compound 20 (990.0 mg, 1.05 mmol), compound 8 (791.6 mg, 1.105 mmol), and HOBt·H₂O (165.7 mg, 1.16 mmol) were dissolved in dichloromethane (6.3 mL) in an argon atmosphere, and then diisopropylethylamine (220 μL, 1.26 mmol) and EDCI·HCl (221.8 mg, 1.16 mmol) were added at 0° C. The resulting mixture was stirred at room temperature for 2 hours, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (dichloromethane/methanol=98:2) to obtain compound 21 (616.9 mg, yield of 36%) as a white amorphous solid. ¹³C NMR (125 MHZ, CDCl₃) δ 173.0, 172.8, 172.7, 172.1, 172.0, 167.8, 156.1, 141.7, 137.2, 130.7, 90.6, 78.7, 71.7, 69.7, 69.1, 58.7, 54., 53.9, 51.7, 41.3, 40.1, 39.0, 39.0, 38.8, 35.7, 34.7, 34.7, 34.7, 32.5, 31.9, 31.1, 29.5, 29.1, 28.8, 28.4, 28.2, 27.2, 27.1, 26.1, 26.0, 23.9, 23.0, 22.8, 19.6, 13.6; HRMS (ESI-TOF): calcd for [C₄₃H₈₁B₁₂IN₁₀O₁₀]²⁻ 577. 3204: found 577. 3209.

Compound 21 (316.5 mg, 0.193 mmol) was dissolved in acetonitrile (3.9 mL) in an argon atmosphere, and then a 1, 4-dioxane solution (4 M, 0.97 mL) of hydrochloric acid was added at 0° C. The resulting mixture was stirred at 0° C. for 1 hour, then a saturated sodium hydrogen carbonate aqueous solution was added to stop the reaction, and the mixture was extracted five times with dichloromethane. The organic layer was washed with saturated brine, dried over magnesium sulfate, and filtered, and then the solvent was removed under reduced pressure to quantitatively obtain compound 22 (336.5 mg) as a white amorphous solid.

HRMS (ESI-TOF): calcd for [C₃₈H₇₃B₁₂IN₁₀O₈]²⁻ 527. 2940: found 527.2937.

### Synthesis of Compound 24 from Compound 22

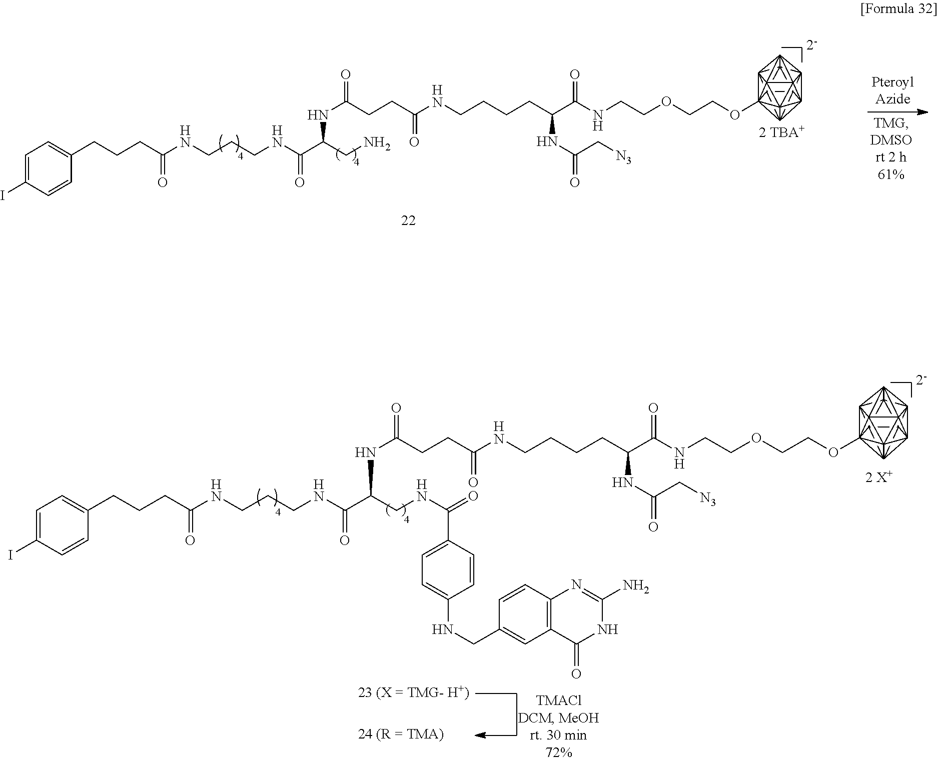

Compound 22 (152.8 mg, 0.105 mmol) and pteroyl azide (33.7 mg, 0.100 mmol) were dissolved in dimethylsulfoxide (1.0 mL) in an argon atmosphere, and then 1, 1, 3,3-tetramethylguanidine (50 μL, 0.400 mmol) was added at room temperature. The resulting mixture was stirred at room temperature for 2 hours, then the mixture was diluted with dichloromethane/methanol, and diethyl ether was added to precipitate the target product. The mixture was filtered, and the resulting yellow solid was washed with acetone and diethyl ether and then dried under reduced pressure to obtain compound 23 (96.5 mg, yield of 61%) as a yellow solid. HRMS (ESI-TOF): calcd for [C₅₂H₈₃B₁₂IN₁₆O₁₀]²⁻ 674. 3376: found 674. 3372.

The obtained yellow solid (92.0 mg, 0.0582 mmol) was dissolved in dichloromethane (1.0 mL) and methanol (1.0 mL), and a methanol solution (1.0 mL) of tetramethylammonium chloride (110.9 mg, 0.582 mmol) was added at room temperature. The resulting mixture was stirred for 30 minutes, then the mixture was filtered, and the resulting orange solid was washed with ethanol/methanol (1:1, 20 mL) and then dried under reduced pressure to obtain compound 24 (63.0 mg, yield of 73%) as a yellow solid.

HRMS (ESI-TOF): calcd for [C₅₂H₈₃B₁₂IN₁₆O₁₀]²⁻ 674. 3376: found 674.3381.

### [Example 4] Synthesis of Molecular Platform BCN-F for PET

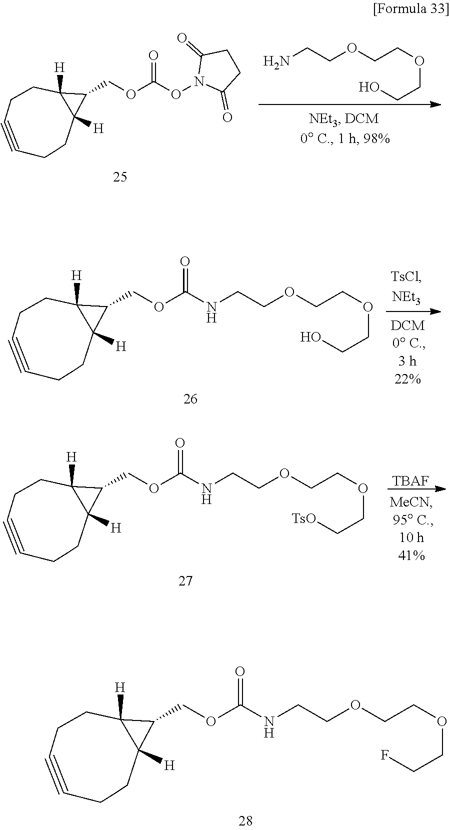

Compound 25 (DeForest, C. A.; Tirrell, D. A. Nat. Mater. 2015, 14, 523) was synthesized according to a known method.

### Synthesis of Compound 26 from Compound 25

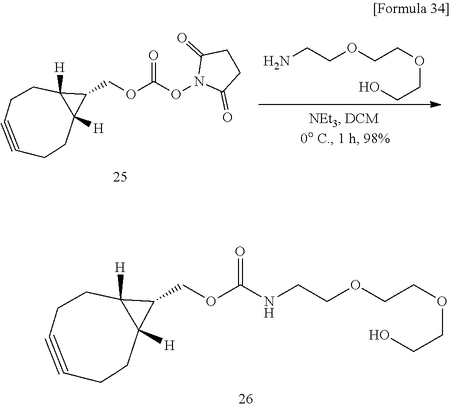 [SMILES: O=C(OC[C@@H]1[C@@H]2CCCC#CCC[C@@H]21)ON1C(=O)CCC1=O ; NCCOCCOCCO ; O=C(NCCOCCOCCO)OC[C@@H]1[C@@H]2CCCC#CCC[C@@H]21]

Compound 25 (190.0 mg, 0.652 mmol) was dissolved in dichloromethane (3.3 mL) in an argon atmosphere, and then triethylamine (178 μL, 1.30 mmol) and triglycolamine (108 μL, 0.783 mmol) were added at 0° C. The resulting mixture was stirred at 0° C. for 1 hour, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (hexane/ethyl acetate=40:60) to obtain compound 26 (207.1 mg, yield of 98%) as a colorless liquid.

¹H NMR (400 MHZ, CDCl₃) δ 5.30 (brs, 1H), 4.16 (d, J=8.1 Hz, 2H), 3.76 (t, J=4.2 Hz, 2H), 3.66-3.52 (m, 9H), 3.39 (q, J=5.1 Hz, 2H), 2.29-2.18 (m, 5H), 1.2-1.57 (m, 2H), 1.41-1.32 (m, 1H), 0.97-0.92 (m, 2H).

### Synthesis of Compound 27 from Compound 26

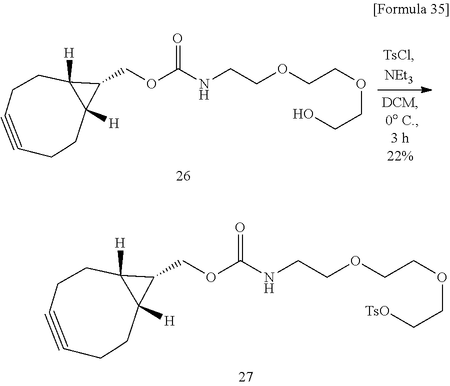 [SMILES: O=C(NCCOCCOCCO)OC[C@@H]1[C@@H]2CCCC#CCC[C@@H]21 ; Cc1ccc(S(=O)(=O)OCCOCCOCCNC(=O)OC[C@@H]2[C@@H]3CCCC#CCC[C@@H]32)cc1]

Compound 26 (98.1 mg, 0.302 mmol) was dissolved in dichloromethane (1.5 mL) in an argon atmosphere, and then triethylamine (84 μL, 0.603 mmol) and tosyl chloride (63.2 mg, 0.332 mmol) were added at 0° C. The resulting mixture was stirred at 0° C. for 3 hours, then a saturated sodium hydrogen carbonate aqueous solution was added to stop the reaction, and the mixture was extracted three times with dichloromethane. The organic layer was washed with saturated brine, dried over magnesium sulfate, and filtered, and then the solvent was removed under reduced pressure. The resulting crude product was purified by silica gel column chromatography (hexane/ethyl acetate=50:50) to obtain compound 27 (31.2 mg, yield of 22%) as a colorless liquid.

¹H NMR (400 MHZ, CDCl₃) δ 7.80 (d, J=8.3 Hz, 2H), 7.34 (d, J=8.3 Hz, 2H), 5.12 (brs, 1H), 4.19-4.11 (m, 4H), 3.71-3.68 (m, 2H), 3.60-3.51 (m, 6H), 3.35 (q, J=5.2 Hz, 2H), 2.45 (s, 3H), 2.32-2.18 (m, 6H), 1.59-1.56 (m, 2H), 1.37-1.33 (m, 1H), 0.96-0.91 (m, 2H).

### Synthesis of Compound 28 from Compound 27

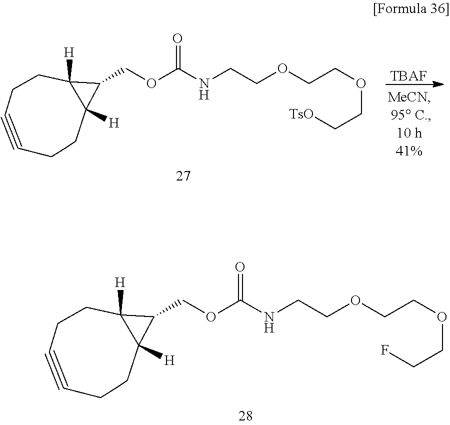

Compound 27 (10.7 mg, 0.0223 mmol) was dissolved in acetonitrile (1.5 mL) in an argon atmosphere, and then a tetrahydrofuran solution (1.0 M, 89 μL) of tetrabutylammonium fluoride was added at room temperature. The resulting mixture was stirred at 95° C. for 10 hours, then a saturated sodium hydrogen carbonate aqueous solution was added to stop the reaction, and the mixture was extracted three times with ethyl acetate. The organic layer was washed with saturated brine, dried over magnesium sulfate, and filtered, and then the solvent was removed under reduced pressure. The resulting crude product was purified by silica gel column chromatography (hexane/ethyl acetate=30:70) to obtain compound 28 (3.0 mg, yield of 41%) as a colorless liquid. ¹H NMR (400 MHZ, CDCl₃) δ 5.15 (brs, 1H), 4.65-4.63 (m, 1H), 4.53-4.51 (m, 1H), 4.15 (d, J=8.0 Hz, 2H), 3.80-3.76 (m, 1H), 3.72-3.63 (m, 5H), 3.57 (t, J=5.3 Hz, 2H), 3.39 (q, J=5.0 Hz, 2H), 2.33-2.17 (m, 6H), 1.63-1.57 (m, 2H), 1.42-1.32 (m, 1H), 0.97-0.92 (m, 2H).

### [Example 5] Synthesis of PBC-IP PET Probe

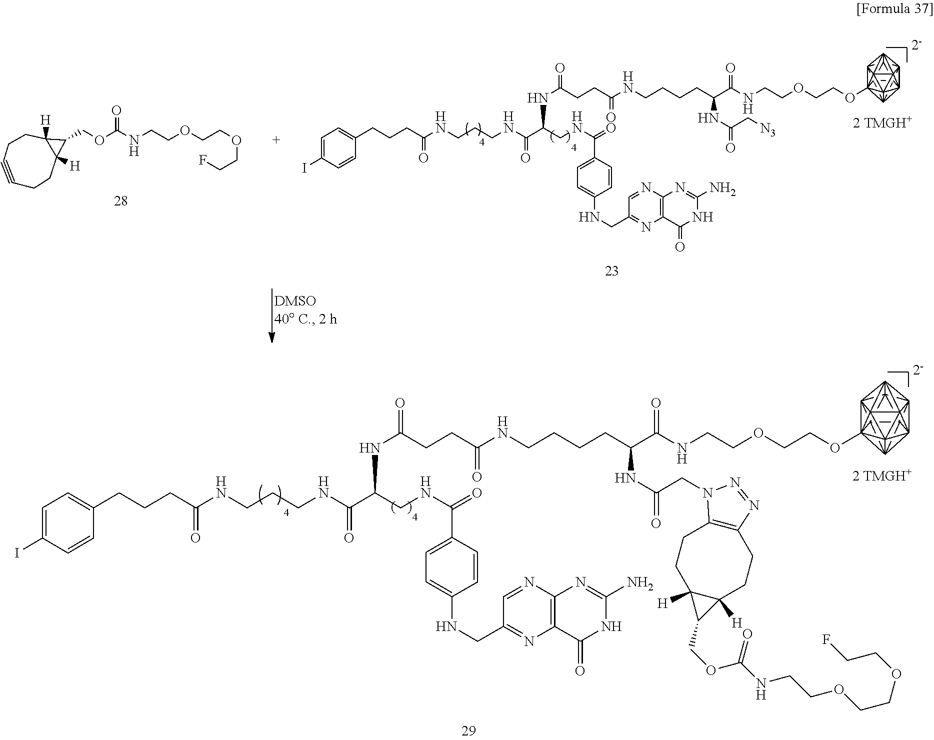

### Synthesis of Compound 29 from Compound 23 and Compound 28

Compound 23 (3.8 mg, 0.00246 mmol) and compound 28 (0.8 mg) were dissolved in dimethylsulfoxide (0.2 mL) in an argon atmosphere. The resulting solution was stirred at 40° C. for 2 hours, and then the solvent was removed by freeze-drying to obtain compound 29 (4.5 mg) as a yellow solid. LRMS (ESI-TOF): calcd for [C₆₉H₁₀₉B₁₂FIN₁₇O₁₄]²⁻ 838.43: found 838.41.

### [Example 6] Evaluation of Binding of PBC-IP to Human Serum Albumin

The ability of PBC-IP to bind to human serum albumin was evaluated by the following method. In addition, for comparison, BC-IP without folic acid and MID (International Publication No. WO 2017/026276), which has been confirmed to covalently bind to albumin, were similarly evaluated for binding ability.

Human serum albumin (HSA, 10 μM, 200 μL) was mixed in phosphate buffered saline (PBS) with a boron compound (MID, PBC-IP, BC-IP) (10 eq, 10 mM, 20μ) in phosphate buffer and reacted at 37° C. for 1 hour. Next, this mixture was washed 9 times (10,000 rpm, 4° C., 5 min) by using a filter (Amicon R Ultra-0.5 mL, 30 K). Finally, the sample was diluted with distilled water to make a total of 5 mL, the boron concentration of the resulting solution was measured by ICP-OES (iCAP 7400 Duo, Thermo), and the affinity evaluation was calculated as the number of bound molecules per HSA molecule.

As a result, it was revealed that 9.0 molecules of PBC-IP bind to 1 molecule of albumin. On the other hand, 2.8 molecules of MID bound thereto, and 3.4 molecules of BC-IP bound thereto, and the numbers of bound molecules were smaller than that of PBC-IP.

### [Example 7] Verification of Folate Receptor (FRα) Expression Level in Each Human Cancer Cell

### (1) Western Blotting Analysis of FR Expression

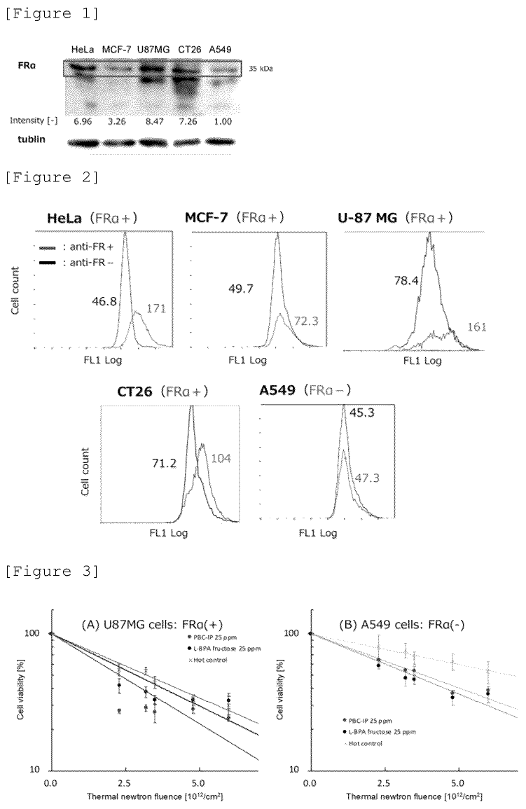

1×10⁶ cells each of HeLa (human cervical cancer), MCF-7 (human breast cancer), U-87 MG (human brain tumor), CT26 (mouse colorectal cancer), and A549 (human lung cancer) cells were added to an SDS-PAGE sample buffer (1 mL) and boiled at 95° C. for 5 minutes, and then the cell lysate was electrophoresed on a 10% SDS polyacrylamide gel and transferred to a PVDF membrane. An anti-FRα antibody (primary antibody) was added, incubated at 4° C. for 12 hours, and then washed three times with TBS buffer. An HRP-conjugated secondary antibody was added, and incubated for further 1 hour at room temperature, then a detection reagent was added, and the protein expression level was detected by chemiluminescence intensity (FIG. 1). As shown in FIG. 1, folate receptors were relatively highly expressed in U87MG cells, HeLa cells, and CT26 cells, whereas the expression level thereof was low in MCF-7 cells and A549 cells.

### (2) Flow Cytometry Analysis of FR Expression

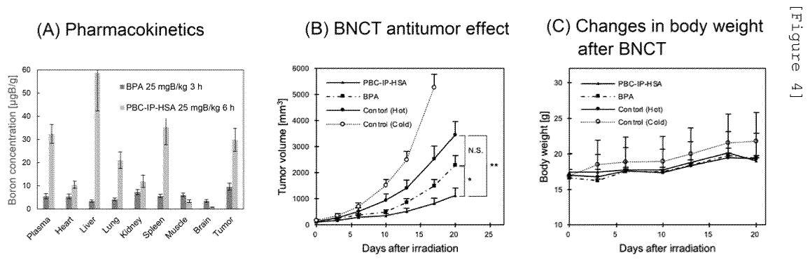

HeLa, MCF-7, U-87 MG, CT26, or A549 cells (5×10⁵ cells) were washed with 1 mL PBS, and an immunoblock solution was added. The resulting solution was incubated at 4° C. for 15 minutes, and then the solution was centrifuged (3 min, 2000 rpm). Next, the supernatant was removed, the primary antibody (20 μg/mL in PBS) was added and incubated at 4° C. for 30 minutes, and then the solution was centrifuged to remove the supernatant. After washing three times with PBS, an FITC-labeled secondary antibody (2 μg/mL in PBS) was added. The resulting solution was incubated at 4° C. for 30 minutes, and then the solution was centrifuged to remove the supernatant followed by washing three times with PBS. Then, the resulting cell pellet was lysed with 500 μL of PBS. Fluorescent signals were observed by using a flow cytometer (FIG. 2). As shown in FIG. 2, the expression of FRα was high in U87MG cells, HeLa cells, and CT26 cells.

### [Example 8] Verification of BNCT Antitumor Effect of PBC-IP (In Vitro)

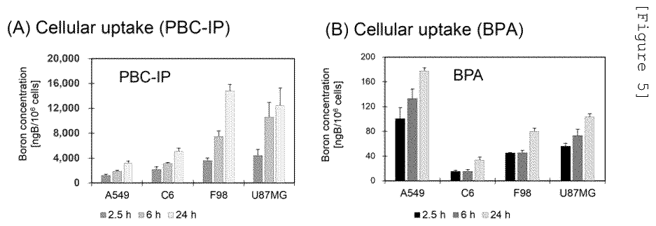

Cells (A549 1000 cells/well or U-87 MG 500 cells/well) seeded in a 96 well plate were cultured together with PBC-IP and L-BPA-fructose at a concentration of 25 ppm [¹⁰B] at 37° C. for 3 hours, and then unilaterally irradiated with thermal neutrons from one side of the plate for 12 minutes. Subsequently, the medium was replaced, the cells were cultured for 96 hours, and then an MTT assay was carried out to evaluate the survival rate of the cells (FIG. 3). The thermal neutron irradiation was carried out by using the irradiation facility of the Institute for Integrated Radiation and Nuclear Science, Kyoto University (KUR), and the cells after irradiation were cultured in the controlled area of KUR. In addition, the survival rate is calculated based on the following expression.

$$Survival rate [ \% ] = \frac{Absorbance ( agent ‐ exposed sample )}{\begin{matrix}Abso rbance ( neutron non ‐ irradiated \\ sample : cold control )\end{matrix}} \qquad [ Expression 1 ]$$

As shown in FIG. 3, PBC-IP dose-dependently exhibited a higher BNCT antitumor effect than BPA on U87MG cells, whereas BPA exhibited a higher BNCT antitumor effect on A549 cells. These results suggest that PBC-IP was taken up via FRα.

### [Example 9] Verification of Pharmacokinetics and BNCT Antitumor Effect of PBC-IP (In Vivo)

Preparation of PBC-IP-HSA: HSA (170.3 mg, 2.56 μmol) and PBC-IP (59.8 mg, 50.9 μmol) were dissolved in PBS (1.8 mL) and stirred at 37° C. for 23 hours. This solution was subjected to 30K ultrafiltration (Amicon Ultra-0.5 mL, Merck Millipore Ltd.), and a PBS solution of the PBC-IP-albumin complex (PBC-IP-HSA) obtained by adjusting the boron concentration to 2500 ppm by ICP-OES measurement was used for the following experiments.

### (A) Pharmacokinetics

U-87 MG cells were subcutaneously implanted into the right thigh of each nude mouse (Balb/cSlc-nu/nu, female, 5 to 6 weeks old, 14 to 20 g), and the mouse was fed with normal solid feed and water and maintained under a 12-hour light/dark cycle in an ambient atmosphere. When the tumor size reached a diameter of 5 to 7 mm, the mouse was injected with 200 μL of a PBS solution of a boron compound (PBC-IP-HSA and BPA: 25 mgB/kg) through the tail vein. Three hours (BPA) and six hours (PBC-IP-HSA) after injection, the mouse was lightly anesthetized, and blood samples were collected by cardiopuncture. Next, the mouse was cervically dislocated and dissected. The liver, the kidney, the spleen, and the tumor were resected, washed with a 0.9% NaCl solution, and weighed. The resected organs were treated with 1 mL of concentrated nitric acid (ultratrace analysis grade) at 90° C. for 2 hours, and then digested samples were diluted with distilled water. The diluted samples were filtered with a hydrophobic filter, and then the boron concentration was measured by ICP-OES (FIG. 4A). As shown in FIG. 4A, PBC-IP-HSA showed more accumulation in the tumor than BPA. In addition, accumulation in the liver and the spleen in addition to the tumor was observed.

### (B) BNCT Antitumor Effect

As described in (A), the U-87 MG tumor-bearing mice were injected with 200 μL of a PBS solution of a boron compound (PBC-IP-HSA, L-BPA: 25 mg (¹⁰B)/kg for each) through the tail vein. The whole mouse was placed in an acrylic mouse holder and fixed to a thermoplastic plate having a thickness of 5 mm. Three hours (L-BPA) or six hours (PBC-IP-HSA) after administration, the right thigh of the mouse was irradiated with a neutron in a dose range of 4.5 to 6.1×10¹² neutrons/cm² in the KUR reactor. The BNCT effect was evaluated based on the changes in tumor volume in the mouse (FIG. 4B). In order to determine the tumor volume, two perpendicular diameters of the tumor were measured with slide calipers and calculated by using the spherical volume expression {4/3π×(R/2)³}. Here, R is the average of the longest dimension and the shortest dimension (each in millimeters) of the tumor. As shown in FIG. 4B, PBC-IP-HSA (25 mg (¹⁰B)/kg, after 6 h) exhibited a significantly high BNCT antitumor effect than L-BPA (25 mg (¹⁰B)/kg, after 3 h).

(C) Changes in Body Weight after BNCT Irradiation

In the irradiation experiment carried out in (B), changes in body weight after irradiation were measured. As shown in FIG. 4C, no significant difference in changes in body weight was observed in either the BPA-irradiated group or the PBC-IP-HSA-irradiated group compared with the control group, indicating that PBC-IP-HSA has sufficiently low toxicity.

### [Example 10] Ability of PBC-IP to Selectively Accumulate in Various Cells

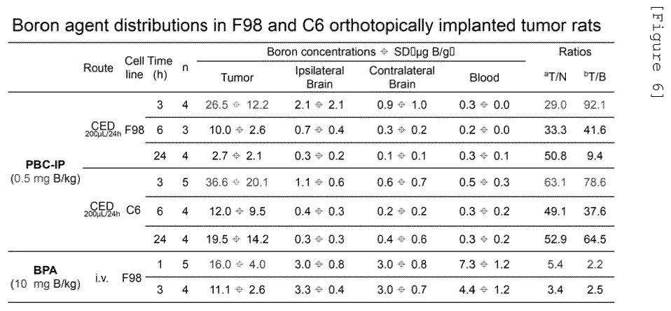

When the accumulation of the PBC-IP complex at the cellular level was compared with that of the existing boron agent (BPA) by using human lung cancer cells (A549), human glioblastoma cells (U87MG), and rat glioblastoma cells (C6, F98), the following result was obtained: PBC-IP was 20 to 150 times better than BPA in terms of cellular accumulation and intracellular retention (FIG. 5). Among these four types of cells, BPA exhibited selective accumulation in the A549 cells in which LAT1 was highly expressed, whereas PBC-IP exhibited selective accumulation in U87MG and F98.

### Experimental Methods

### (Cell)

F98 rat malignant glioblastoma cells were provided by Dr. Rolf Barth (Department of Pathology, The Ohio State University, Columbus, OH, US) and were cultured at 37° C. in 5% CO² by using Dulbecco's modified Eagle's medium (DMEM) supplemented with 10% fetal bovine serum (FBS) and 10% penicillin/streptomycin/amphotericin B. The F98 rat malignant glioblastoma cells were histologically characterized as anaplastic astrocytoma. All materials for cell culture were purchased from Gibco Invitrogen Corporation (Grand Island, NY).

### (Intracellular Boron Accumulation Concentration)

First, 5×10⁵ cells were placed in a 100 mm dish (Becton Dickinson, Franklin Lakes, New Jersey) and cultured at 37° C. in 5% CO₂ by using the medium described above. The cells were cultured for 72 hours, then the medium was replaced with a medium containing 5 μg B/mL of BPA, BSH, or PBC-IP, and the cells were cultured for 2.5 hours, 6 hours, and 24 hours. After that, the medium containing the boron compound was removed, and the cells were washed twice with 4% phosphate buffered saline (PBS) and then detached with a trypsin-ethylenediaminetetraacetic acid solution. After that, a medium was added, centrifugation (200×g, 5 minutes) was carried out twice, and cells were counted and finally sedimented. After that, the cells were digested with a 1 N nitric acid solution (Wako Pure Chemical Industries, Osaka Japan) overnight, and the amount of boron taken up was measured by inductively coupled plasma-atomic emission spectroscopy (ICP-AES) using an iCAP6300 emission spectrometer (Hitachi High-Technologies, Tokyo, Japan).

### [Example 11] BNCT Therapeutic Effect of PBC-IP on F98 and C6 Malignant Glioblastoma Cell-Implanted Rat Brain Tumor Models

The optimum administration conditions by intravenous administration were studied in an F98 rat malignant glioblastoma model. When PBC-IP was administered at a high concentration or a high dose, it was confirmed that the blood boron concentration was as high as 150 ppm or more (about 20 to 30 ppm for BPA), and in order to ensure safety, it was determined that an effect could be expected by administration at about 1/10 of the above concentration or dose in treatment. Further, in order to determine the optimum dose, a dose escalation test was carried out in the lower dose range, and it was found that the toxicity of the present agent tends to depend on the concentration rather than the dose of the administered agent. In local administration using Convection Enhanced Delivery (CED), stably high intratumoral boron accumulation at about 25 ppm has been confirmed, and the accumulation ratio is 30 times or more based on accumulation in the normal brain, and the blood concentration is extremely low, and thus the concentration ratio is about 100 times.

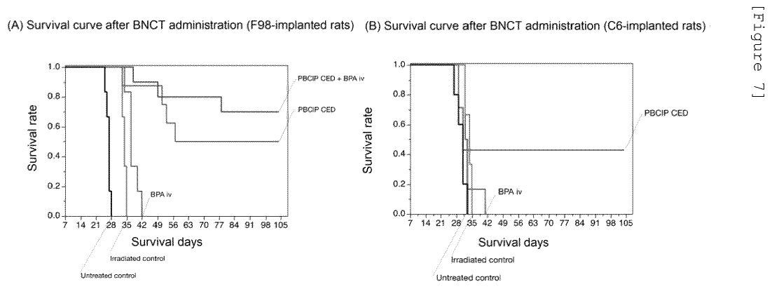

The optimum administration conditions by local administration (CED) of PBC-IP were studied by using F98 and C6-implanted rat brain tumor models (FIG. 6). For intravenous administration of BPA (10 mgB/kg), one hour after administration, the F98 tumor boron concentration was about 16 ppm, the normal brain tissue (T/N) ratio was 5.4, and the blood concentration ratio (T/B) was 2.2. On the other hand, when PBC-IP was administered at a low dose (0.5 mgB/kg), the F98 tumor boron concentration was about 26.5 ppm 3 hours after administration of PBC-IP (CED), the normal brain tissue (T/N) ratio was 29, and the blood concentration ratio (T/B) was 92, which are extremely high values. Similarly, the C6 tumor boron concentration reached 36.6 ppm, the normal brain tissue (T/N) ratio was 63, and the blood concentration ratio (T/B) was 78, which are high values.

Three hours after administration of PBC-IP (CED), neutron irradiation was carried out to verify the therapeutic effect of neutron capture therapy (FIG. 7). The brain tumor models were divided into an untreated group, a neutron irradiation only group, a BNCT with BPA (iv) group, a BNCT with local administration of PBC-IP (CED) group, and a PBC-IP (CED) plus BPA (iv) group to carry out a treatment experiment, and the survival times after tumor implantation were compared. The therapeutic effect of intravenous BPA as a control for BNCT treatment represented a significant prolongation of survival time with a median survival time of 37 days. In contrast, the survival time prolonging effect is extremely high in the PBC-IP (CED) group, and more than half of the subjects survived even at an observation point of 90 days, and the median value was unable to be calculated, which is a good result. The effect was even higher in the group that received treatment with PBC-IP (CED) alone plus intravenous BPA, indicating the antitumor effect of PBC-IP on a BPA-refractory tumor site. In the C6-implanted rat brain tumor model, the tumor boron concentration by PBC-IP (CED) was about 36 ppm, which was higher than that for F98. Neutron irradiation experiments in this model are limited to a small number of pilot studies, but long-term survival of the rats was obtained only with PBC-IP (CED).

Neutron irradiation was carried out after local administration (CED) of the PBC-IP complex into the rat brain, and the influence of BNCT using the same agent on normal brain tissue was pathologically studied. There was no obvious physical or neurological influence during the post-irradiation observation period, and in the pathological study of a brain tissue after neutron irradiation, no obvious tissue adverse event was observed other than a mild tissue reaction localized only to the insertion site of a catheter used for brain local administration (CED) and the surroundings thereof (FIG. 8).

### Experimental Methods

### (F98 Brain Tumor Orthotopic Model Rat)

All male Fischer rats (F344 Japan SLC; Hamamatsu, Shizuoka, Japan) having a body weight of 200 to 240 g were anesthetized by intraperitoneal injection of a mixed anesthetic including the three types of anesthetics: medetomidine (0.4 mg/kg), midazolam (2.0 mg/kg), and butorphanol (5.0 mg/kg). The rats were then fixed in a stereotaxic frame (Model 900; David Kopf Instruments, Tujunga, California). Subsequently, a midline scalp incision was made, and a 1 mm burr hole was drilled 1 mm posterior and 4 mm right lateral to the bregma by using an electric drill. A 25 μL Hamilton syringe with a 26 gauge needle (model 1700RN; Hamilton Bonaduz, Switzerland) was inserted into the rat brain to implant F98 tumor cells. The injection needle was first inserted to a depth of 6 mm from the dura mater and then withdrawn to a distance of 1 mm to the intracerebral target (5 mm from the dura mater). A F98 cell suspension diluted with 10 μL of DMEM containing 1.4% agarose was infused at a rate of 20 μL/min by using an automatic infusion pump in such a way as to achieve a concentration of 10³ cells for a treatment experiment and 10⁵ cells for a biodistribution experiment. After infusion, the needle was immediately withdrawn, the burr hole was covered with bone wax, and the scalp was sutured.

### (CED Method)

CED is a direct drug administration method that can locally infuse a drug into the interstitium of the brain under sustained low positive pressure to obtain a high concentration and a broad distribution of the drug (Yin et al. Cancer Gene Ther. 2013, 20:336-341; Bobo et al. Proc. Natl. Acad. Sci. USA, 1994, 91:2076-2080). For drug CED, an Alzet osmotic pump (model #2001D; DURECT Corporation, Cupertino, California) and an intracerebral infusion kit (rigid stainless-steel cannula, 5-mm 28 gauge) were assembled and filled with 200 μL of a boron compound solution. F98 malignant glioblastoma cell-bearing rats were anesthetized, and then an infusion pump was subcutaneously implanted in the back of the rat. A needle connected to an infusion cannula was inserted into the same burr hole through which the tumor cells were implanted. This infusion pump was able to administer a boron compound at a rate of 8 μL/h for 24 hours or more.

### (Boron Concentration in Each Tissue)

Fourteen days after tumor implantation, boron compounds (BPA and PBC-IP) were administered to the F98 malignant glioblastoma cell-bearing rats. The biodistributions 2 and 6 hours after intravenous administration of 12 mg B/kg body mass (b.m.) of BPA, and 2, 6, and 24 hours after the end of PBC-IP (CED) administration were measured by using 3 to 5 rats per group. Rats were euthanized after each administration, and the tumor, the normal brain, the blood, the heart, the lung, the liver, the spleen, the kidney, the skin, and the muscle thereof were excised and weighed, and then digested with a 1 N nitric acid solution. The boron concentration (μg B/g) in each organ was measured by ICP-AES.

### (Neutron Irradiation Experiment)

In a BNCT experiment, 14 days after implantation of 10³ F98 malignant glioblastoma cells, neutron irradiation was carried out by using KURRI. Malignant glioblastoma cell-bearing rats were randomly divided into 5 groups (groups 1 to 5) so that each group consisted of 6 to 10 rats. Group 1 is an untreated control group, and was transported to the reactor and anesthetized, and handled in the same manner as the animals in the other groups except that no neutron irradiation (sham irradiation) was carried out. Group 2 is a neutron-irradiated control group. Group 3 is a group that received intravenous administration of BPA and then neutron irradiation. Group 4 is a group that received administration of PBC-IP (¹⁰B-enrich) by CED and then neutron irradiation. Group 5 is a group (combination group) that received administration of PBC-IP (¹⁰B-enrich) by CED plus intravenous injection of BPA and then neutron irradiation. The rats were anesthetized with a mixed anesthetic, and then the whole body except the head was shielded and fixed on a board. Two hours after the end of intravenous administration or 3 hours after the end of CED, the rats were irradiated with a neutron beam having a reactor output of 5 MW for 20 minutes. After neutron irradiation, neutron non-irradiated animals and neutron-irradiated animals were left at KURRI for observation. The therapeutic effect was evaluated by the survival times of all rats.

All publications, patents, and patent applications cited herein are incorporated herein by reference.

### INDUSTRIAL APPLICABILITY

The boron agent of the present invention is used as pharmaceuticals, and thus the present invention is industrially applicable to industries related to pharmaceuticals.

1. A boron agent for boron neutron capture therapy, comprising a compound represented by the following formula (I) or (II): 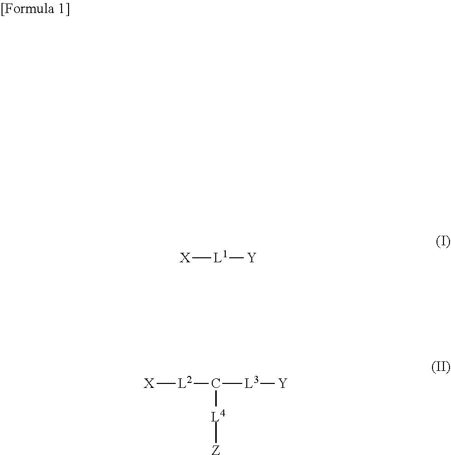
wherein C represents a carbon atom, L¹, L², L³, and L⁴ each independently represent a divalent group that functions as a spacer, X represents a group that binds to albumin, Y represents a group containing ¹⁰B, and Z represents a group that binds to a folate receptor.

2. The boron agent for boron neutron capture therapy according to claim 1, wherein L¹, L², L³, and L⁴ in formulas (I) and (II) are each an alkylene group, provided that one or more —CH₂— of the alkylene group may optionally be substituted with —O—, —S—, —NH—, or —CO—.

3. The boron agent for boron neutron capture therapy according to claim 1 or 2, wherein X in formulas (I) and (II) is a group represented by any of the following formulas (A) to (C): 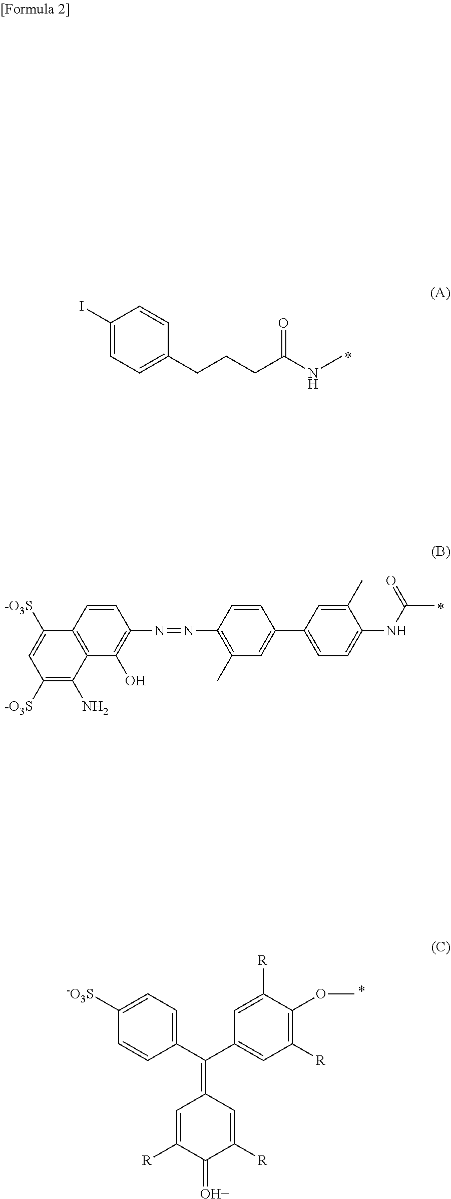
wherein * represents a point of attachment, and R represents a hydrogen atom, a fluorine atom, a chlorine atom, a bromine atom, or an iodine atom.

4. The boron agent for boron neutron capture therapy according to any one of claims 1 to 3, wherein Y in formulas (I) and (II) is a group derived from a boron cluster.

5. The boron agent for boron neutron capture therapy according to any one of claims 1 to 4, wherein Z in formula (II) is a group represented by the following formula (D): 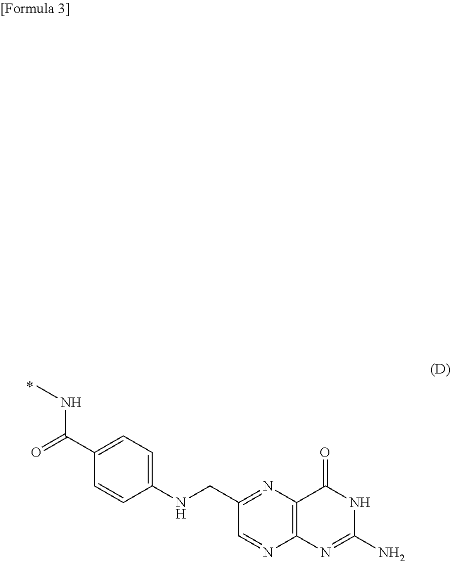
wherein * represents a point of attachment.

6. The boron agent for boron neutron capture therapy according to claim 1, wherein the compounds represented by formulas (I) and (II) are compounds represented by the following formulas (Ia) and (IIa), respectively:

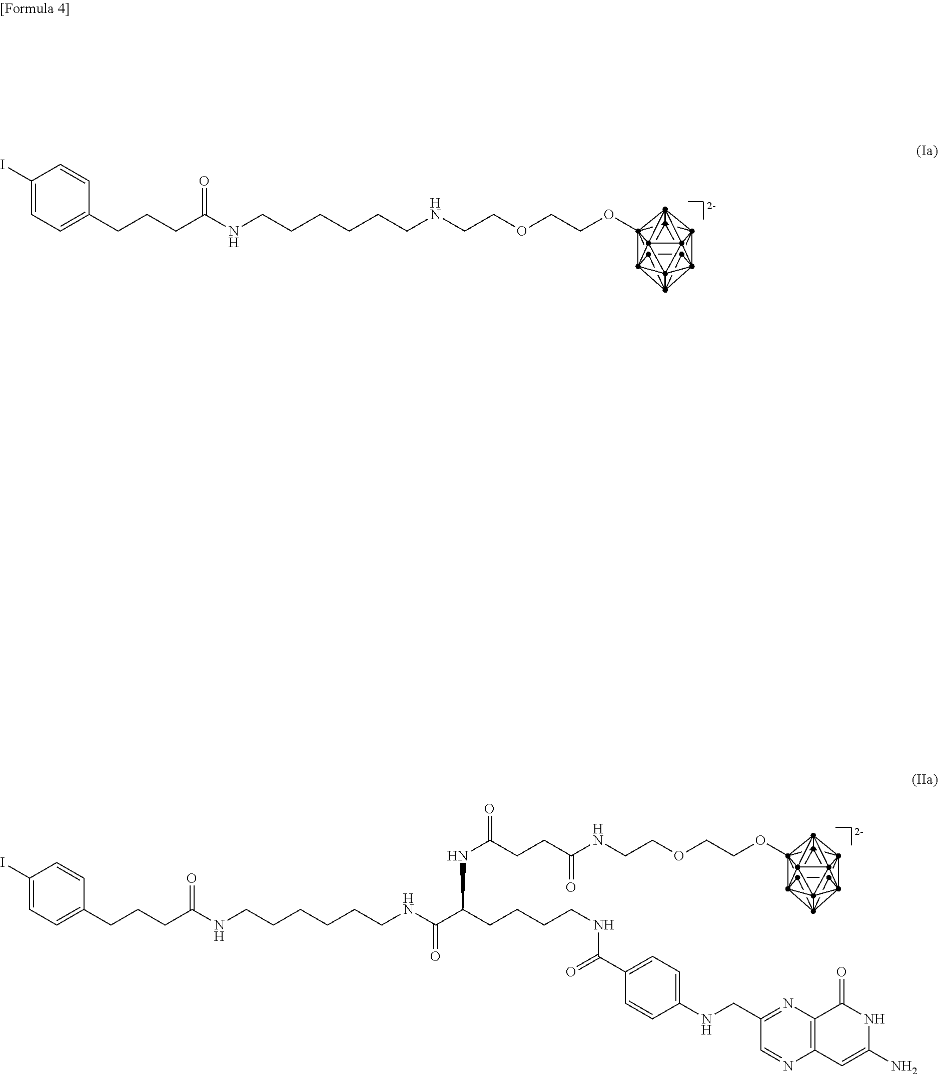

7. An imaging agent for PET, comprising a compound represented by the following formula (III) or (IV): 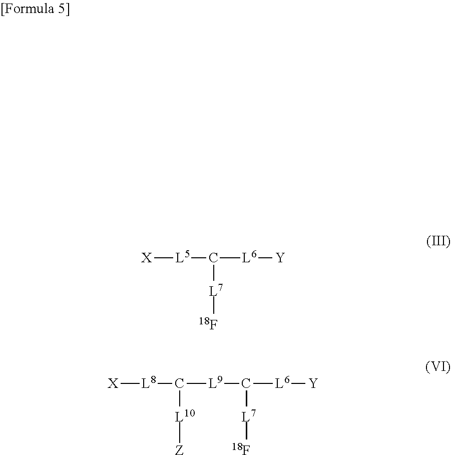
wherein C represents a carbon atom, ¹⁸F represents a radioactive fluorine atom having a mass number of 18, L⁵, L⁶, L⁷, L⁸, L′, and L¹⁰ each independently represent a divalent group that functions as a spacer, X represents a group that binds to albumin, Y represents a group containing ¹⁰B, and Z represents a group that binds to a folate receptor.

8. The imaging agent for PET according to claim 7, wherein L⁵, L⁶, L⁸, L⁹, and L¹⁰ in formulas (III) and (IV) are each an alkylene group, provided that one or more —CH₂— of the alkylene group may optionally be substituted with —O—, —S—, —NH—, or —CO—, and L⁷ is an alkylene group, provided that one or more —CH₂— of the alkylene group may optionally be substituted with —O—, —S—, —NH—, or —CO—, and one —CH₂— of the alkylene group may optionally be substituted with a divalent group formed by a click reaction.

9. The imaging agent for PET according to claim 7 or 8, wherein X in formulas (III) and (IV) is a group represented by any of the following formulas (A) to (C): 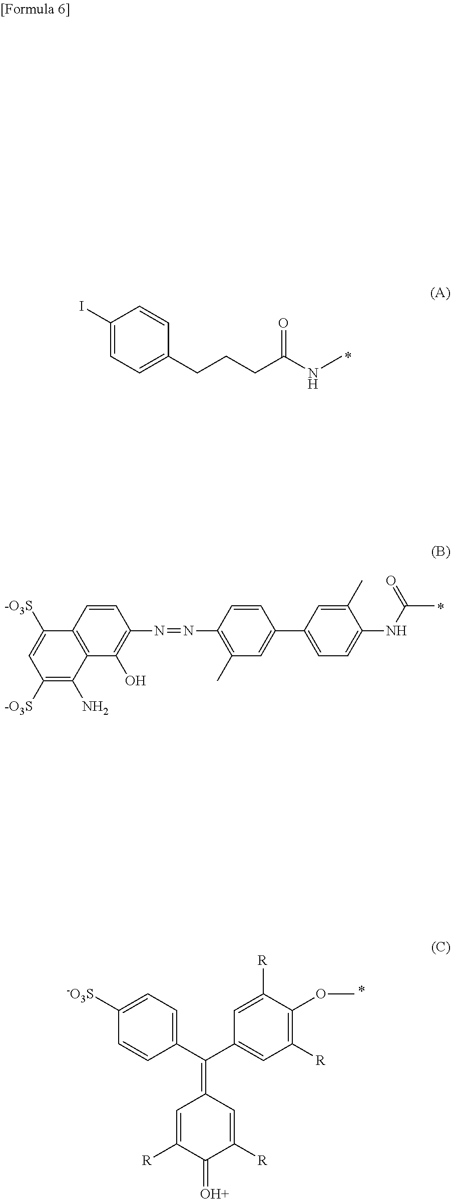
wherein * represents a point of attachment, and R represents a hydrogen atom, a fluorine atom, a chlorine atom, a bromine atom, or an iodine atom.

10. The imaging agent for PET according to any one of claims 7 to 9, wherein Y in formulas (III) and (IV) is a group derived from a boron cluster.

11. The imaging agent for PET according to any one of claims 7 to 10, wherein Z in formula (IV) is a group represented by the following formula (D): 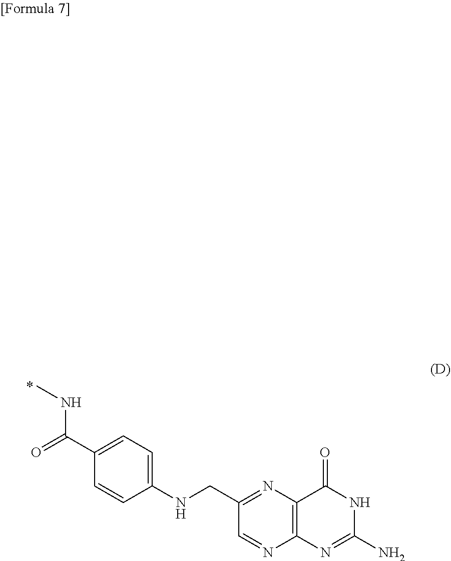
wherein * represents a point of attachment.

12. The imaging agent for PET according to claim 7, wherein the compound represented by formula (IV) is a compound represented by the following formula (IVa): [chemical structure]
